(19) United States
(12) Patent Application Publication
(10) Pub. No.: US 2026/0306768 A1
(43) Pub. Date: Oct. 8, 2026

(54) SYSTEM AND METHOD FOR AIRWAY MUSCLE ACTIVATION USING AIR PRESSURE ROUTINES

(71) Applicant: NOVARESP TECHNOLOGIES INC., Halifax (CA)

(72) Inventors: Meagan Mackenzie Sinclair, Halifax (CA); Hamed Hanafialamdari, Halifax (CA); Richard Lee Balys, Bedford (CA); Robin Bernard LeBlanc, Dartmouth (CA); Gregory Eric Avard Begin, Halifax (CA); Guillermo Aristi Suarez, Halifax (CA)

(21) Appl. No.: 19/166,146

(22) PCT Filed: Apr. 5, 2024
(86) PCT No.: PCT/CA2024/050445

§ 371 Date: Sep. 17, 2025

Related U.S. Application Data
(60) Provisional application No. 63/457,747, filed on Apr. 6, 2023.

(51) Int. Cl. A61M 16/00

(52) U.S. Cl. CPC A61M 16/0006; A61M 16/0009; A61M 16/026; A61M 2016/0039; A61M 2016/0042; A61M 2205/502; A61M 2205/52

(57) ABSTRACT

Various embodiments are described herein for a method for stimulating one or more airway muscles of a user of a breathing assistance device. The method may be performed by a processor and comprises: receiving sensor data from at least the user; processing the sensor data to predict whether a respiratory failure event is imminent or to detect that the respiratory event is currently occurring when the user is using the breathing assistance device; selecting one or more of air pressure routines to prevent the predicted respiratory failure event or to mitigate the respiratory failure event that is currently occurring; adjusting the control signal of the breathing assistance device based on the selected one or more pressure routines; and providing the control signal to the breathing assistance device to apply the selected one or more air pressure routines to the user.

### CROSS-REFERENCE TO RELATED APPLICATION

This application claims priority from U.S. Provisional Patent Application No. 63/457,747 filed Apr. 6, 2023, the entire contents of which is hereby incorporated herein by reference in its entirety.

### FIELD

The various embodiments described herein generally relate to muscle activation using air pressure routines. More specifically, the various embodiments relate to applying a pressure routine to a user of a breathing assistance device to activate the airway muscles of the user and provide breathing assistance during certain apnea events.

### INTRODUCTION

Individuals suffering acute or chronic respiratory (Chronic Obstructive Pulmonary Disease (COPD), asthma, Acute Respiratory Distress Syndrome (ARDS)) or respiratory-related conditions (e.g., sleep apnea) may require assistive devices to maintain respiratory functions at normal levels. Assistive devices such as mechanical ventilators, Positive Airway Pressure (PAP) devices or Continuous Positive Airway Pressure (CPAP) devices are common to provide breathing assistance. However, while such assistive devices are critical with respect to maintaining normal respiratory functions, these devices may also cause harm and distress to a user as a result of the stress or strain due to the amount of pressure or flow imparted on the user's respiratory system. Moreover, these devices are mostly reactive and not proactive to predict and prevent respiratory distress or discomfort. As such, there is a desire for methods and systems to identify and minimize user harm.

It is known in the art that there are various levels of mechanical support for different sorts of respiratory failure. In the most basic form, the inspired concentration of oxygen may be increased to percentages above 21% which is the normal atmospheric content of oxygen. This helps a patient in need to satisfy the metabolic need of oxygen for their body.

The next higher level of ventilatory support addresses the problem of when the oxygen content of the inhaled gas mixture is not sufficient to keep the homeostasis of the patient's body. This means that also retention of CO2 is becoming a problem. For these sorts of respiratory failures, a more invasive way of ventilation including active elevation of the airway pressure above the atmospheric pressure is involved to eliminate CO2 as the end product of the metabolism of the body. As this involves a tightly fitting mask, there are limits to the pressure that can be applied to the system.

If elevation of inhaled oxygen and increase of airway pressure facilitated by the mask is no longer sufficient then so called mechanical ventilation using an intratracheal tightly fitted tube and/or tracheostomy along with a ventilator is used for ventilation. The parameters that are controlled with a ventilator include the volume of each breath applied to the patient, the respiratory rate per minute which, when taken together, allow for the volume of ventilation of the patient to be controlled along certain time intervals, e.g., every minute. In addition, typically mechanical ventilation also controls for the fraction of inhaled oxygen from 21% to 100% in air and the inspiratory to expiratory ratio of the breathing cycle. If these measures are not sufficient to keep blood oxygen and CO2 levels within safe physiological limits then opposed end expiratory pressure (PEEP) and an I/E inspiratory to expiratory ratio is applied. As far as monitoring of ventilation is concerned there are a variety of methods known in the art that include end tidal CO2, inspired CO2, inspired O2, expired O2, blood gas analysis of arterial blood pressure/volume diagrams and volumetric measures of the inspired and expired volumes of ventilation in the patient.

Sleep apnea includes obstructive apnea (OA) which occurs when the airway closes and obstructs flow while a user continues respiratory effort [1]. OAs occur with a collapse of the airway due to decrease in muscle tone of the genioglossus muscle [2], [3]. The flow can be partially obstructed resulting in an obstructive hypopnea or a flow limitation. Studies have found that airways of patients with Obstructive Sleep Apnea (OSA) typically contain anatomical irregularities that need higher muscle activation levels to stay open [5]. In contrast, a central apnea (CA) is a pause in flow caused by lack of respiratory effort while the airway remains open [4].

To address this and reduce the number of apneas, the use of neurostimulators has been investigated. These tools use electrical stimulation to activate the hypoglossal nerve whose motoneurons are the main controller of the upper airway muscles. This can activate the upper airway muscles and keep the throat from collapsing and therefore prevent OAs [3], [6], [7]. Studies have concluded that number of OAs can be reduced with electrical stimulation of the upper airway muscles continuously throughout the night [8].

For CA, transvenous phrenic nerve pacing (remedē®, Respicardia, Inc.) has emerged as an entirely novel therapy for CSA. It restores physiologic nocturnal breathing via an implanted device without the use of positive pressure ventilation. Utilizing technology similar to pacemakers and neurostimulators, this device utilizes transvenous leads placed in the chest to detect apneas. The leads deliver electricity to directly stimulate the phrenic nerve and restore normal movement of the diaphragm.6

However, the use of embedded devices may be quite invasive, and the application of electrical stimulation may not be tolerated by certain users or users may have trouble sleeping when the electrical stimulation is used. Accordingly, there is a need to apply a different type of stimulus that is effective at activating the upper airway muscles and keep the throat from collapsing and therefore quickly adjust the treatment to the user to prevent or quickly stop a central or obstructive sleep apnea event.

### SUMMARY OF VARIOUS EMBODIMENTS

According to one broad aspect of the teachings herein, in at least one embodiment described herein there is provided a method for stimulating one or more airway muscles of a user of a breathing assistance device, wherein the method is performed by a processor, and the method includes: (a) receiving sensor data from at least the user; (b) processing the sensor data to predict whether a respiratory failure event is imminent or to detect that the respiratory event is currently occurring when the user is using the breathing assistance device; (c) selecting one or more of the air pressure routines to prevent the predicted respiratory failure event or to mitigate the respiratory failure event that is currently occurring; (d) adjusting the control signal of the breathing assistance device based on the selected one or more pressure routines; and (e) providing the control signal to the breathing assistance device to apply the selected one or more air pressure routines to the user.

In at least one embodiment, the one or more air pressure routines includes varying between different slopes of applied pressure magnitude over time.

In at least one embodiment, the one or more air pressure routines includes combinations of negative and positive pressure pulses.

In at least one embodiment, the one or more air pressure routines includes repeating pressure pulses.

In at least one embodiment, the one or more air pressure routines includes combinations of negative and positive pressure spikes.

In at least one embodiment, the one or more air pressure routines includes repeating pressure spikes of varying amplitude.

In at least one embodiment, the one or more air pressure routines includes combinations of sinusoidal pressure waves with differing properties.

In at least one embodiment, the one or more air pressure routines includes combinations of sinusoidal pressure waves and pressure spikes or pulses.

In at least one embodiment, the method may further include: receiving subsequent sensor data from at least the user subsequent to the application of the selected one or more air pressure routines; processing the subsequent sensor data to predict whether a respiratory failure event remains imminent when the user is using the breathing assistance device; adjusting the control signal of the breathing assistance device to apply the selected air pressure routine at a higher amplitude; and providing the control signal to the breathing assistance device to apply the selected one or more air pressure routines to the user at the higher amplitude.

According to another broad aspect of the teachings herein, in at least one embodiment described herein there is provided a controller for controlling the operation of a breathing assistance device for stimulating one or more airway muscles of a user that uses the breathing assistance, wherein the controller includes: an input for receiving sensor data from at least the user; a memory unit that comprises instructions for applying one or more air pressure routines during operation of the breathing assistance device; a processor that is electronically coupled to the input to receive the sensor data, the processor being configured to generate a control signal for controlling the breathing assistance device by: processing the sensor data to predict whether a respiratory failure event is imminent or is detect that the respiratory event is currently occurring when the user is using the breathing assistance device; selecting one or more of the air pressure routines to prevent the predicted respiratory failure event or to mitigate the respiratory failure event that is currently occurring; adjusting the control signal of the breathing assistance device based on the selected one or more pressure routines; and providing the control signal to the breathing assistance device to apply the selected one or more air pressure routines to the user.

In at least one embodiment, the controller is further configured to perform a method based on any of the embodiments described in accordance with the teachings herein.

According to one broad aspect of the teachings herein, in at least one embodiment described herein there is a system for stimulating the airway muscles of a user of a breathing assistance device, wherein the system includes: a breathing assistance device that generates an airflow to the user; an entry element that is coupled to the breathing assistance device and is worn by the user to provide the airflow to the user during use; and a breathing assistance device controller that is defined based on any of the embodiments described in accordance with the teachings herein.

In at least one embodiment, the breathing assistance device comprises a high flow delivery device.

In at least one embodiment, the breathing assistance device comprises a gas delivery device including an oxygen delivery device.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 is a block diagram of an example embodiment of a breathing assistance system for controlling or tuning a breathing assistance device during use by a user.

FIG. 2 is a block diagram of another example embodiment of a breathing assistance system for controlling or tuning a breathing assistance device during use by a user by applying different air pressure routines in accordance with the teachings herein.

FIG. 3A shows an example of a multi-slope variable pressure routine in accordance with the teachings herein.

FIG. 3B shows an example of mask pressure and flow rate graphs from the application of the pressure routine of FIG. 3A.

FIG. 4 shows an example of a combination of negative and positive pressure pulses routine in accordance with the teachings herein.

FIG. 5 shows an example of a repeating pressure pulses routine in accordance with the teachings herein.

FIG. 6A shows an example of a combined negative and positive pressure spikes routine in accordance with the teachings herein.

FIG. 6B shows an example of mask pressure and flow rate graphs from the application of the pressure routine of FIG. 6A.

FIG. 7 shows an example of repeating variable amplitude pressure spikes routine in accordance with the teachings herein.

FIG. 8A shows an example of a combined pressure sinusoids routine in accordance with the teachings herein.

FIG. 8B shows an example of mask pressure and flow rate graphs from the application of the pressure routine of FIG. 8A.

FIG. 9A shows an example of a combined pressure sinusoids and pulses or spikes routine in accordance with the teachings herein.

FIG. 9B shows an example of mask pressure and flow rate graphs from the application of the pressure routine of FIG. 9A.

FIG. 10 is a flowchart of an example embodiment of a method for choosing and applying a pressure routine to a user of a breathing assistance device.

FIG. 11A shows an example of electromyography (EMG), flow, pressure and raw intramuscular EMG graphs from the application of a pressure spike.

FIG. 11B shows an example mean filtered EMG graph from the application of ten pressure spikes.

FIG. 11C shows an example maximum filtered EMG graph from the application of ten pressure spikes.

FIG. 11D shows an example ensemble average pressure and filtered EMG graph from the application of ten pressure spikes.

FIG. 11E shows an example of EMG, flow, pressure and abdominal effort graphs from the application of a pressure spike.

FIG. 11F shows an example mean filtered EMG graph from normal inspiration and normal inspiration with a pressure spike.

FIG. 11G shows an example maximum filtered EMG graph from normal inspiration and normal inspiration with a pressure spike.

FIG. 11H shows an example mean filtered EMG graph from the application of forty pressure spikes.

FIG. 11I shows an example maximum filtered EMG graph from the application of forty pressure spikes.

FIG. 11J shows an example ensemble average pressure and filtered EMG graph from the application of forty pressure spikes.

FIG. 12 shows an example of EMG, flow, pressure and raw intramuscular EMG graphs from the application of a repeating pressure pulse routine.

FIG. 13 shows another example of EMG, flow, pressure and raw intramuscular EMG graphs from the application of a repeating pressure pulse routine.

FIG. 14 shows another example of EMG, flow, pressure and raw intramuscular EMG graphs from the application of a pressure spike.

FIG. 15 shows another example of EMG, flow, pressure and raw intramuscular EMG graphs from the application of a pressure spike.

FIG. 16 shows another example of EMG, flow, pressure and raw intramuscular EMG graphs from the application of a combination sinusoidal routine.

FIG. 17 shows an example of filtered EMG, flow, pressure and raw intramuscular EMG versus time graphs demonstrating the application of a repeating pressure spike routine.

FIG. 18 shows another example of filtered EMG, flow, pressure and raw intramuscular EMG versus time graphs demonstrating the application of a repeating pressure spike routine.

FIG. 19 shows an example of filtered EMG, flow, pressure and raw intramuscular EMG versus time graphs demonstrating the application of a negative pressure spike.

FIG. 20 shows another example of filtered EMG, flow, pressure and raw intramuscular EMG versus time graphs demonstrating the application of a positive pressure spike.

FIG. 21 shows an example of filtered EMG, flow, pressure and raw intramuscular EMG versus time graphs demonstrating the application of a combination negative and positive pressure spike routine.

FIG. 22 shows an example of filtered EMG, flow, pressure and raw intramuscular EMG versus time graphs demonstrating the application of a combination sinusoidal pressure waves and negative and positive pressure spike routine.

FIG. 23 is a flowchart of an example embodiment of a method for choosing and applying a pressure routine to a user of a breathing assistance device.

Further aspects and features of the example embodiments described herein will appear from the following description taken together with the accompanying drawings.

### DETAILED DESCRIPTION OF THE EMBODIMENTS

Various embodiments in accordance with the teachings herein will be described below to provide an example of at least one embodiment of the claimed subject matter. No embodiment described herein limits any claimed subject matter. The claimed subject matter is not limited to devices or methods having all of the features of any one of the devices or methods described below or to features common to multiple or all of the devices and or methods described herein. It is possible that there may be a device or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

It will be appreciated that for simplicity and clarity of illustration, where considered appropriate, reference numerals may be repeated among the figures to indicate corresponding or analogous elements or steps. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, the terms coupled or coupling can have a mechanical, fluidic or electrical connotation. For example, as used herein, the terms coupled or coupling can indicate that two elements or devices can be directly connected to one another or connected to one another through one or more intermediate elements or devices via an electric signal, an electrical connection, a mechanical element, a fluid or a fluid transport pathway, for example, depending on the particular context.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof. As another example, the phrase “A, B, C or any operable combination thereof” is mean to cover any combination of elements A, B and C that provides utility which may, for example, include A, B, C, A and B, A and C, B and C, or A, B and C.

It should be noted that terms of degree such as “substantially”, “about” and “approximately” as used herein mean a reasonable amount of deviation of the modified term such that the end result is not significantly changed. These terms of degree may also be construed as including a deviation of the modified term such as, but not limited to, 1%, 2%, 5% or 10%, if this deviation would not negate the meaning of the term it modifies.

Furthermore, the recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as, but not limited to, 1%, 2%, 5% or 10%, for example.

At least a portion of the example embodiments of the devices, systems or methods described in accordance with the teachings herein may be implemented as a combination of hardware and software. For example, at least a portion of the embodiments described herein may be implemented, at least in part, by using one or more computer programs, executing on one or more programmable devices comprising at least one processing element and at least one storage element (i.e., at least one volatile memory element and at least one non-volatile memory element). The hardware may comprise one or more input devices including, but not limited to, one sensor, a touch screen, a keyboard, a mouse, buttons, keys, sliders or any combination thereof, as well as one or more output devices including, but not limited to, an output port for transmitting a control signal, a display, a speaker, a printer, or any combination thereof depending on the implementation of the hardware.

It should also be noted that there may be some elements that are used to implement at least part of the embodiments described herein that may be implemented via software that is written in a high-level procedural language such as object oriented programming. The program code may be written in C++, C #, JavaScript, Python, or any other suitable programming language and may comprise modules or classes, as is known to those skilled in object-oriented programming. Alternatively, or in addition thereto, some of these elements implemented via software may be written in assembly language, machine language, or firmware as needed. In either case, the language may be a compiled or interpreted language.

At least some of these software programs may be stored on a computer readable medium that is readable by a general or special purpose programmable device. The software program code, when read by the programmable device, configures the programmable device to operate in a new, specific and predefined manner in order to perform at least one of the methods described herein. Examples of computer readable medium include, but are not limited to, a ROM, a magnetic disk, an optical disc, a USB key, and the like that is readable by a device having a processor, an operating system, and the associated hardware and software that is necessary to implement the functionality of at least one of the embodiments described herein. The software program code, when read by the device, configures the device to operate in a new, specific, and predefined manner (e.g., as a specific-purpose computer) in order to perform at least one of the methods described herein.

At least some of the programs associated with the devices, systems, and methods of the embodiments described herein may be capable of being distributed in a computer program product comprising a computer readable medium that bears computer usable instructions, such as program code, for one or more processors. The program code may be preinstalled and embedded during manufacture and/or may be later installed as an update for an already deployed computing system. The medium may be provided in various forms, including non-transitory forms such as, but not limited to, one or more diskettes, compact disks, tapes, chips, and magnetic and electronic storage. In alternative embodiments, the medium may be transitory in nature such as, but not limited to, wire-line transmissions, satellite transmissions, internet transmissions (e.g., downloads), media, digital and analog signals, and the like. The computer useable instructions may also be in various formats, including compiled and non-compiled code.

It should be noted that the term “user” covers a person who is using a breathing assistance device. In some cases, the user may be an individual that is using the breathing assistance device in their home or a non-medical setting. In other cases, the user may be a patient who is using the breathing assistance device in a medical setting such as a clinic or a hospital, for example.

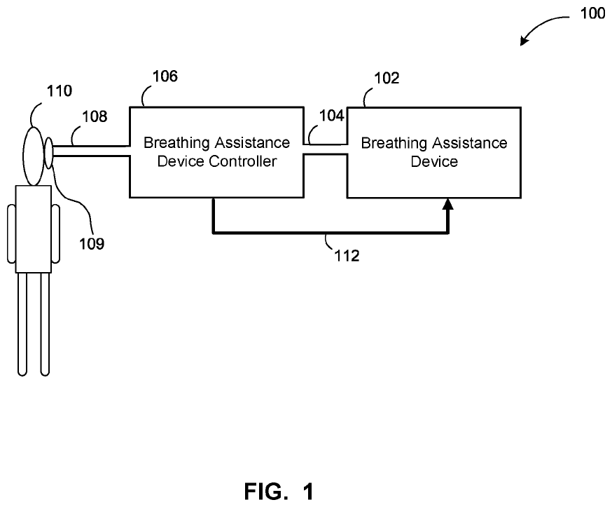

Referring now to FIG. 1, illustrated therein is a block diagram of a breathing assistance system 100 for applying a pressure routine to a user in accordance with at least one embodiment of the teachings herein. In at least one embodiment, the breathing assistance system 100 can also be used for controlling or tuning a breathing assistance device using the forced oscillation technique based on detection and/or prediction of respiratory failure which may be performed in various ways such as, but not limited to, the techniques described in issued U.S. Pat. No. 11,413,414B2 entitled “METHOD AND APPARATUS FOR BREATHING ASSISTANCE”, U.S. Pat. No. 11,633,560B2 entitled “METHOD AND APPARATUS FOR CONTINUOUS MANAGEMENT OF AIRWAY PRESSURE FOR DETECTION AND/OR PREDICTION OF RESPIRATORY FAILURE”, U.S. Pat. No. 11,612,708B2 entitled “METHOD & APPARATUS FOR DETERMINING AND/OR PREDICTING SLEEP AND RESPIRATORY BEHAVIOURS FOR MANAGEMENT OF AIRWAY PRESSURE” and/or the Applicant's PCT patent application no. PCT/CA 2024/050324 filed on Mar. 18, 2024 entitled “SYSTEM and METHOD for GENERATING A PERSONALIZED PREDICTIVE MODEL FOR MANAGEMENT OF AIRWAY PRESSURE”, which are each hereby incorporated by reference in their entirety.

The system 100 comprises a breathing assistance device 102 that generates an airflow that is provided to a user 110 via air transport pathways 104 and 108 and, for example, a laryngeal tube, a breathing mask or an endotracheal tube 109 (hereinafter collectively referred to as an “entry element”). The airflow can be at least one pressure pulse of air, a continuous flow of air, or a superposition of pressure pulses of air and a continuous flow of air. The airflow is controllable by adjusting at least one of the air pressure and flow rate of the breathing assistance device 102 via corresponding input controls on the breathing assistance device 102.

In at least one embodiment, the breathing assistance device 102 may be a mechanical ventilator for providing breathing support to the user. Alternatively, in at least one embodiment, the breathing assistance device 102 may be a CPAP, APAP, BiPAP or PAP device for providing breathing support to the user. In another alternative, in at least one embodiment, the breathing assistance device 102 may be a respiratory treatment delivery device such as, but not limited to, respiratory treatment delivery devices that assist a user in clearing their lungs and coughing out secretions. In another alternative, in at least one embodiment, the breathing assistance device comprises a high flow delivery device. Alternatively, in at least one embodiment, the breathing assistance device comprises a gas delivery device including an oxygen delivery device. In other instances, the breathing assistance device 102 may be an anesthesia machine in the OR, an ICU ventilator, a home ventilator and oxygenator of COPD, and any other machine that provides breathing assistance to a user who has a respiratory disease. Therefore, in general, pressure routines provided in accordance with the teachings herein to reduce, remove or pre-empt respiratory failure can be used with all types of ventilation including invasive (with tube) and non-invasive (tubeless) ventilation.

A breathing assistance device controller 106 is coupled to the breathing assistance device 102 via the air transport pathway 104 (which may also be called the flow passage 104) and receives airflow from the breathing assistance device 102 and delivers the airflow via the air transport pathway 108 and the entry element 109 to the user 110. It should be noted that the term “air” in the present disclosure is used generally to denote the flow of gas and other airborne particles through the system 100. For example, the output of a mechanical ventilator may include gasses and/or vapors other than air such as, but not limited to, anesthetics, for example which are typically vapors but can also be gases. In a PAP device, water vapor may be combined with air. In some embodiments of the breathing assistance device 102, gaseous medication (i.e., steroids, oxygen, Nitrogen, etc.) may be added to the air flow and provided to the patient under ventilation based on respiratory health and/or measured comfort level. For example, the medication may include an appropriate amount of steroids that may be used daily to improve the CPAP experience for the user. The airflow may be delivered to the user 110 via the entry element 109. In the present example embodiment, the entry element 109 may be a mask worn over the user's 110 nose and mouth, just over the nose or adjacent or inside the nostrils of the user 110 for alternative masks. In other embodiments, the entry element 109 may be an endotracheal tube inserted into the trachea by means of intubation or tracheostomy.

In embodiments in which the breathing device 102 is a mechanical ventilator, there are actually two air pathways (not shown) instead of just the air transport pathway 104 (which may also be called a flow passage) where one of the pathways is used for inhalation and the other of these pathways is used for exhalation. The pathways shown in FIG. 1 apply for the case where the breathing assistance device 102 is a PAP device. It may be thus understood that the breathing assistance device 102 provides at least one pathway to allow air to flow from the air transport pathway 104 to the air transport pathway 108. It may further be understood that there can be embodiments in which the breathing assistance device controller 106 is at least partially or completely incorporated “inline” with the airflow pathways from the breathing assistance device 102 to the user 110.

In the present example embodiment, the breathing assistance device controller 106 may comprise one or more sensors (not shown) to measure various parameters of the airflow being delivered to the user 110. For example, sensors can be attached to the mask worn by the user 110 which may result in improved SNR for the sensor data obtained from the sensors. Alternatively, the sensors, such as ultrasonic sensors for example, can be attached in the tubing pathway. In either case, these sensors can be used to measure airflow parameters associated with both inspiration and expiration. However, in the case of a PAP machine, such sensors are located close to the mask because the tube 108 only carries an inspiratory flow whereas in a mechanical ventilator the sensors can be attached to the mask or endotracheal tube or they can be located anywhere along the tubes that are used for the inspiratory pathway and the expiratory pathway.

In some embodiments, the breathing assistance device controller 106 may not include these sensors but may instead read these parameters from the breathing assistance device 102 since the breathing assistance device 102 may also be equipped with sensors for measuring airflow parameters. In at least one embodiment, the breathing assistance device controller 106 may further comprise a device to provide a forced oscillation signal, in order to provide changes in air pressure for the airflow provided to the user 110. In some embodiments, a sensor for measuring both air pressure and airflow is present. In other embodiments, dedicated sensors may be used to measure the airflow or the air pressure such that more than one sensor may be used with the breathing assistance device controller 106. For example, some sensor technologies use a laser to detect movement or ultrasound can be used to detect both pressure and flow rate using one sensor (as the measured flow rate can be determined from dividing the measured pressure by a known resistance).

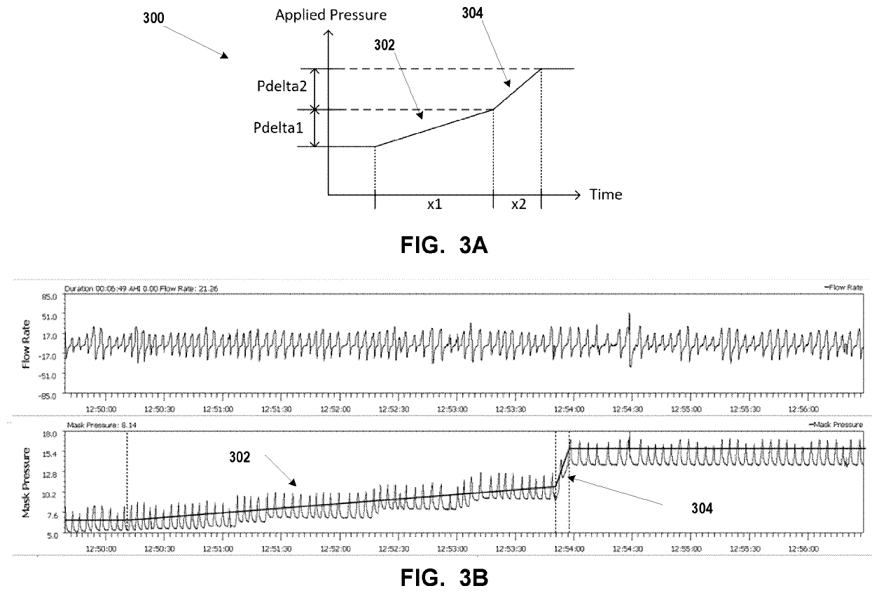

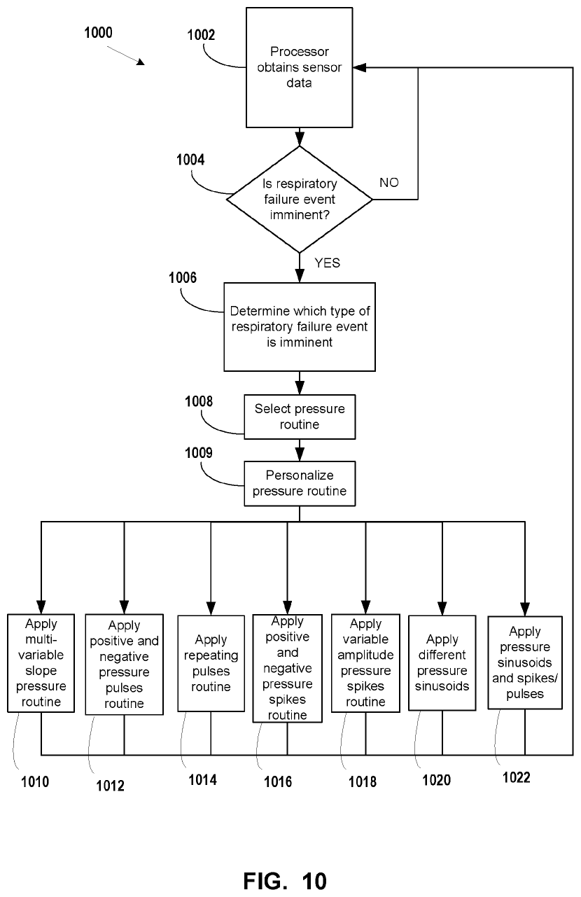

The measured airflow parameters such as air volume, air pressure and airflow rate may be used by the breathing assistance device controller 106 to generate a control signal 112 that can be used as feedback to adjust the operation of the breathing assistance device 102. For example, in some embodiments there can be methods that, for example, perform detection and/or prediction of when a respiratory failure event occurs/will occur and then sends a control signal 112 to the breathing assistance device to proactively apply an appropriate pressure routine such as, but not limited to, those described further below in relation to FIGS. 3A-9B, for example, to the user so that the user does not experience the detected/predicted respiratory failure event. This pressure routine may be determined in accordance with a method such as that shown in FIG. 10, for example.

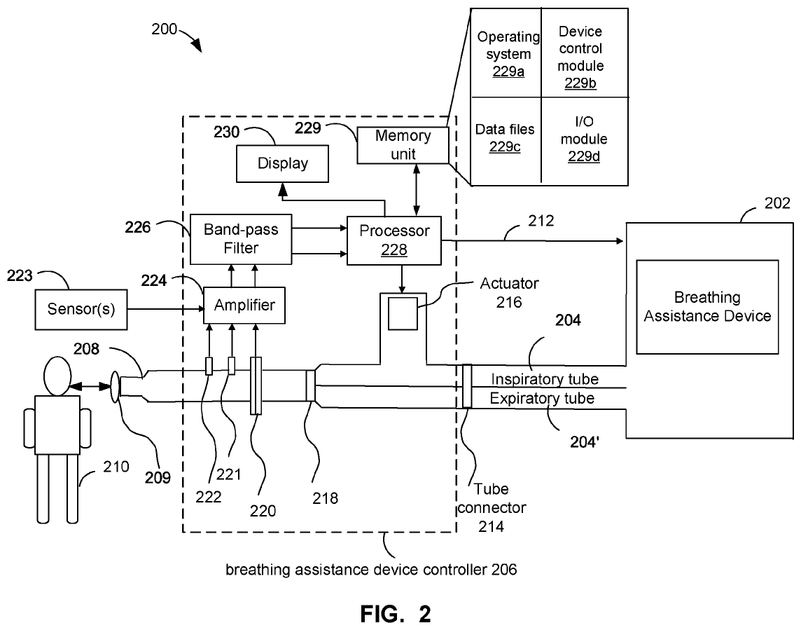

Referring now to FIG. 2, shown therein is a block diagram of an example embodiment of a breathing assistance system 200 for controlling or tuning a breathing assistance device 202 during use by a user 210 by applying a pressure routine to the user, in accordance with the teachings herein. Elements in FIG. 2 that correspond to elements in FIG. 1 have been numbered similarly. Similar to the configuration of the breathing assistance system 100, a breathing assistance device 202 generates airflow that is provided to a user 210 via air transport pathways 204 and 204′ and the breathing tube 208 and the airflow is monitored by a breathing assistance device controller 206 for modifying the operation of the breathing assistance device 202 under certain conditions. Similar to FIG. 1, the airflow may be delivered to the user 210 via an entry element 209. In this example embodiment, the entry element 209 may be a mask worn over to fluidically couple with the user's 210 nose (i.e., nostrils) and optionally the user's mouth. In other embodiments, the entry element 209 may be an endotracheal tube inserted into the trachea by means of intubation or tracheostomy.

FIG. 2 provides additional details with respect to the various system components that may be employed. In some embodiments, the breathing assistance device 202 may be a mechanical ventilator for providing breathing support. In other embodiments, the breathing assistance device 202 may be a PAP device for providing breathing support. Other options are available for the breathing assistance device 202 as explained previously for the breathing assistance device 102.

In the present embodiment, the breathing assistance device 202 is a mechanical ventilator and includes an inspiratory tube 204 and an expiratory tube 204′ for providing an airflow pathway for airflow leaving and returning to the breathing assistance device 202, respectively. The inspiratory tube 204 and the expiratory tube 204′ may be connected to the breathing assistance device controller 206 at one airflow pathway using the tube connector 214. The airflow may then flow to the user 210 through another airflow pathway of the breathing assistance device controller 206.

In at least one embodiment, the airflow from the inspiratory tube 204 may be subjected to perturbation from a forced oscillation produced by a motor or an actuator (hereinafter referred to as an “actuator” to refer to both cases) 216 generating an oscillation of air at a desired frequency and intensity. The actuator 216 may be one of a loud speaker, an electromagnet, a piezoelectric device, a piston and a motor, for example. The choice of actuator may be dependent on the design specifications such as the physical size of the device 206 as well as on the limitations imposed on the Bill of Materials (BOM). It should be noted that in some embodiments, the actuator 216 can be included in the breathing assistance device 202 and not in the breathing assistance device controller 206.

Alternatively, in some embodiments, both the breathing assistance device 202 and the breathing assistance device controller 206 can include separate actuators.

In at least one embodiment in which FOT may be used, the oscillation pressure signal has an oscillation frequency that may be at any frequency that is practical for performing air pressure and airflow measurements during FOT measurement. For example, one of the FOT methods described in U.S. Pat. Nos. 11,413,414B2, 11,633,560B2, 11,612,708B2, and/or PCT patent application no. PCT/CA2024/050324 may be used.

The inspiratory tube 204 and the expiratory tube 204′ may be combined prior to reaching the user 210 at a junction using a tube fitting 218 connected to a breathing tube 208. Subsequent to the tube fitting 218, the combined airflow may be sensed to determine airflow parameters such as the airflow rate and the air pressure. In this example embodiment, one or more sensors are used including, but not limited to, a flow transducer 220 and a pressure transducer 221. It should be noted that the flow transducer 220 may also be called a flow rate transducer or an airflow transducer. The sensor type used for the transducers 220 and 221 can be any appropriate transducer device, including but not limited to, ultrasonic, pneumatic or piezoelectric transducers, for example. In some embodiments, the airflow parameters can be measured and calculated by recording the pressure drop across a pneumotachograph, which is used as the sensor.

The outputs of the flow transducer 220 and the pressure transducer 221 may be preconditioned prior to being further processed and analyzed. For example, the output signals from the transducers 220 and 221 may be amplified by an appropriate amplifier 224 to obtain the desired signal amplitudes. For example, in some embodiments, the amplifier 224 may be a lock-in amplifier which may be used to reduce signal noise to help focus on the frequency of interest. It should be noted that separate amplifiers can be used for each measured signal or a dual channel amplifier may be used.

The amplified signal may then be filtered to remove extraneous frequency domain information. In the present embodiment, a band-pass filter 226 with a tuned center-frequency corresponding to the frequency of the oscillation produced by the actuator 216 may be used. In some embodiments, the passband may be made sufficiently narrow such that a notch filter can be used when a single frequency is used for FOT. The band-pass filter 226 has a narrow passband but it is preferably large enough to contain any side lobes in the measured signals that contain modulated breathing information.

After the signals have been amplified and filtered, the signals are received by the processor 228 for further processing and analysis which may include determining when to apply one or more pressure routines described herein as well as select which of the one or more pressure routines to apply and then generate a control signal 212 that is provided to the breathing assistance device 202 to adjust its operation in accordance with the selected one or more pressure routines, as described in more detail below.

In some embodiments, the processor 228 may a programmable device such as a programmable microcontroller or a field programmable gate array (FPGA). In other embodiments, the processor 228 may be part of a single-board computer system platform such as the Arduino platform, or Raspberry Pi platform. In yet other embodiments, there may be more than one processor which is used when significant data requires processing. In yet other embodiments, the signal filtering may be performed using the processor 228 by using digital signal processing (DSP) techniques such that a separate filtering device 226 may not be necessary.

The control signal 212 can be provided to the breathing assistance device 202 using any method known to those skilled in the art. For example, the control signal 212 can be provided through a wired connection. However, in other implementations, the control signal may be communicated wirelessly to the breathing assistance device 202 in which case the system 200 can include a transmitter or a transceiver (such as a WiFi or Bluetooth transceiver).

The measured airflow parameters, indices and/or control signal 212 may also be shown on an optional display 230 provided on the breathing assistance device controller 206. The display 146 may be, but is not limited to, an LCD display such as that for a tablet device, smartphone or the breathing assistance device 202.

The system 200 also includes a memory unit 229 which can include RAM, ROM, one or more hard drives, one or more flash drives or some other suitable data storage elements such as disk drives, etc. The memory unit 229 stores program instructions for an operating system 229a, a device control module 229b, one or more data files 229c, and an input/output (I/O) module 229d. In at least one embodiment, there may also be a machine learning module along with machine learning models that may be used for detecting and/or predicting a respiratory failure event. Other software instructions may be included as is known to those skilled in the art.

A respiratory failure event can include any event that deviates from normal breathing. For example, in sleep apnea related breathing, a respiratory failure event may include a flow limitation, snoring, obstructive apnea, central apnea, obstructive hypopnea, central hypopnea, respiratory effort related arousals (RERA), and/or arousals. As another example, in users with diseases such as chronic obstructive pulmonary disease, a respiratory failure event may include stiffening of the lung, exacerbation, and/or flow limitation. As another example, in users using an ICU ventilator, a respiratory failure event may include failing of weaning of the user from ventilation, pneumonia, and/or resistive or elastic events of the lung. Other respiratory failure events may be determined.

The device control module 229b comprises software instructions that, when executed, configures the processor 228 to operate in a particular manner to implement various functions, processes, and methods for the system 200. For example, the device control module 229b can include program instructions for receiving various sensed/measured data from the sensors 223, performing various analysis/processing using the sensed data and then generating a control signal based on the analysis/processing to control the operation of the breathing assistance device 202 to provide appropriate airflow to the user. The processing may include the detection and/or prediction of a respiratory failure event, which may be done using various techniques such as one or more of those described in U.S. Pat. Nos. 11,413,414B2, 11,633,560B2, 11,612,708B2, and/or PCT Patent Application No. PCT/CA2024/050324. In accordance with the teachings herein, the device control module 229b can include program instructions to select one or more pressure routines described herein and generate a control signal based on the one or more selected pressure routine to provide airflow to the user that is defined based on the selected one or more pressure routines. In at least one embodiment, the one or more pressure routines may be selected based on the type and/or severity of the detected or predicted respiratory event.

The data used to develop, test, and validate the methods described herein was obtained at the QEII Health Sciences Centre in Halifax. The data included thousands of sleep apneas of various categories including normal breathing, flow limitation, respiratory effort related arousals (RERAs), obstructive apnea, central apnea and obstructive hypopnea, central hypopnea and other sleep apnea respiratory events.

The input/output module 229d receives input data that was obtained by the sensors or received from electronic communications with another computing device and/or sends outputs data to the computing device or signals such as control signal 212 from processing done by the device control module 229b that are then sent to the corresponding hardware. The communication may be provided by a cellular radio, a BlueTooth radio, a network adaptor or a computer port like a USB port (all not shown). The input/output module 229d may, for example, operate in conjunction with the device control module 229b to communicate data (or signals) between one or more of the processor 228 and one or more of the sensors 220 to 223 and the display 230. The functionality of the input/output module 229d may be implemented, for example, using a combination of hardware, firmware, and/or software.

The data files 229c may store any temporary data (e.g., data that is not needed after the breathing assistance device 202 has been used or permanent data (e.g., data saved for later use), such as user data (e.g., a user ID), settings for the breathing assistance device 202, and preprocessing or other processing settings including variables and calibration data. The data files 229c may also include various user data for each user that uses the breathing assistance device 202 such as identification data, respiratory physiological data and recorded user data during use of the breathing assistance device 202.

In at least one embodiment, the breathing assistance device controller 206 can be configured to operate continuously to monitor the pressure and flowrate of the airflow provided to the user 210 to allow for detecting and/or predicting the user's breathing state for constant adjustment of the operation of the breathing assistance device 202. Doing so may permit real-time or near real-time adaptive adjustments to be made to minimize or avoid any respiratory failure event from being experienced by the user 210. In other embodiments, the breathing assistance device controller 206 may alternatively be controlled to operate intermittently, for example, at a set time interval such as every 30 seconds or every 60 seconds. Such operating conditions may be preferred if the breathing assistance device controller 206 is battery operated so as to help extend the operational lifetime of the breathing assistance device 202.

The sensors may also include at least two additional sensors 222 and 223. Sensor 222 can be a CO2 gas sensor. The sensors 223 can include one or more user sensors that may be placed at certain locations on the user 210. For example, sensors 223, which may be optional, can include sensors that are used to obtain at least one physiological signal and/or at least one neurological signal. For example, the physiological signals include one or more of ECG, EOG, and EMG signals and the neurological signals include one or more of EEG and Peripheral Neurophysiological Examination (PNE) signals. These signals can be measured using known electrodes that are placed at certain locations on the user 210 as is known by those skilled in the art. As the position of a user can affect the therapy provided by the breathing assistance device, sensors 223 can additionally include positional sensors placed at certain locations on the user 210 to determine the position of the user. The CO2 signal is also a physiological measurement that can be obtained. Sensors 223 can additionally include environmental sensors, placed at different locations within the room where the breathing assistance device 202 is used and sensors placed on or within the breathing assistance device 202 to measure properties of the breathing assistance device 202. These sensors can be used to for example, monitor the health of physical components of the breathing assistance device. For example, a humidity sensor may be placed within the breathing assistance device 202 to monitor a humidity level of the device. The sensor data may be pre-processed as is known by different channels of the amplifier 224 and the band-pass filter 226 to reduce noise for these particular signals before these signals are sent to the processor 228 for further analysis. The settings for the amplification and filtering of the signals are known to those skilled in the art. The sensors 223 that are used depends on the algorithms used to detect and/or predict a respiratory failure event where these algorithms are described in U.S. Pat. Nos. 11,413,414B2, 11,633,560B2, 11,612,708B2, and/or PCT Patent Application No. PCT/CA2024/050324.

FIGS. 3A-9B show various examples of pressure routines that may be applied to the user of the breathing assistance device to proactively avoid the user from experiencing a respiratory failure event or reducing the effect of a currently occurring respiratory failure event. In general, the air pressure routines described herein comprise at least two sequential waveforms that have different shapes or similar shapes with different characteristics. The application of one or more pressure routines described herein to a user may stimulate one or more nerves in the human body that are associated with respiratory muscles and cause the activation of one or more of the respiratory muscles, thereby preventing a respiratory failure event from occurring or reducing the effects of a currently occurring respiratory failure event. Respiratory muscles may include one or more airway muscles from the nose to the diaphragm through the respiratory system. In at least one embodiment, each pressure routine may be applied to the user for a certain period, then return to a baseline pressure. In other embodiments, two or more pressure routines may be applied consecutively and in varying orders of occurrence.

FIGS. 3A-3B show a multi-variable pressure slope routine 300. This routine involves varying between different slopes of applied pressure magnitude over time. FIG. 3A is a graph of applied pressure to the user versus time. For a first period of time, a first pressure slope 302 is applied to the user (i.e., the applied pressure is increased at a given constant rate over the first time period). Subsequently, a second pressure slope 304 is applied to the user. In the embodiment shown in FIG. 3A, the second pressure slope 304 is greater than the first pressure slope 302. In other embodiments, the second pressure slope 304 is less than the first pressure slope 302. In the embodiment shown in FIG. 3A, the multi-variable pressure routine 300 involves applying two different pressure slopes consecutively. In other embodiments, the multi-variable pressure routine 300 may involve applying any number of different pressure slopes consecutively, which may depend on the severity of the expected or currently occurring respiratory failure event.

FIG. 3B shows example flow rate and mask pressure versus time graphs demonstrating the application of the multi-variable pressure slope routine 300 on normal breathing with a PAP machine. The first pressure slope 302 is applied to the user followed by the second pressure slope 304. A stable flow rate, amplitude, and pattern (or in other words, normal breathing) is maintained during and after the application of the pressure routine to the user.

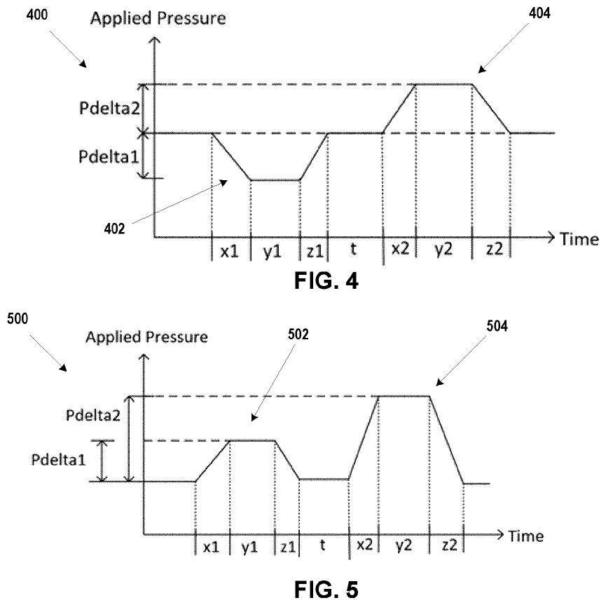

FIG. 4 shows a combination of negative and positive pressure pulses routine 400. This routine involves varying between negative and positive pressure pulses. FIG. 4 is a graph of applied pressure to the user versus time. In the embodiment shown in FIG. 4, a negative pressure pulse 402 is first applied to the user. A negative pressure pulse, such as the negative pressure pulse 402, includes applying pressure with a negative slope for a period of time, subsequently keeping pressure approximately constant for a period of time, and then subsequently applying pressure with a positive slope for a period of time to reach the initial pressure. In some embodiments, such as the example shown in FIG. 4, after the negative pressure pulse 402 is applied, the pressure may be held approximately constant for a period of time. In the example embodiment, a positive pressure pulse may then be applied after the negative pressure pulse. FIG. 4 shows the application of a positive pressure pulse 404 following the pressure being held approximately constant. A positive pressure pulse, such as the positive pressure pulse 404, includes applying pressure with a positive slope for a period of time, subsequently keeping pressure approximately constant for a period of time, and then subsequently applying pressure with a negative slope for a period of time to reach the initial pressure. In other embodiments, the routine 400 may involve applying a positive pressure pulse followed by a negative pressure pulse, with or without holding an approximate constant pressure in between. In the embodiment shown in FIG. 4, the pressure routine 400 involves applying two pressure pulses consecutively. In other embodiments, the routine 400 may involve applying any number of pressure pulses consecutively, varying their amplitudes and/or varying the amount of time t in between successive pulses, which may depend on the severity of the expected or currently occurring respiratory failure event.

FIG. 5 shows a repeating pressure pulses routine 500. FIG. 5 is a graph of applied pressure to the user versus time. The pressure routine 500 includes applying consecutive positive pulses, similar to positive pressure pulse 404, or consecutive negative pulses, similar to negative pressure pulse 402. FIG. 5 shows an example embodiment of a first positive pressure pulse 502 followed by a second positive pressure pulse 504. The second positive pressure pulse 504 reaches a higher maximum pressure than the first positive pressure pulse 502. In other embodiments, the pressure pulses may all reach the same maximum pressure or different maximum pressures. Other embodiments may also involve applying more than two pressure pulses consecutively, varying their amplitudes and/or varying the amount of time t in between successive pulses, which may depend on the severity of the expected or currently occurring respiratory failure event.

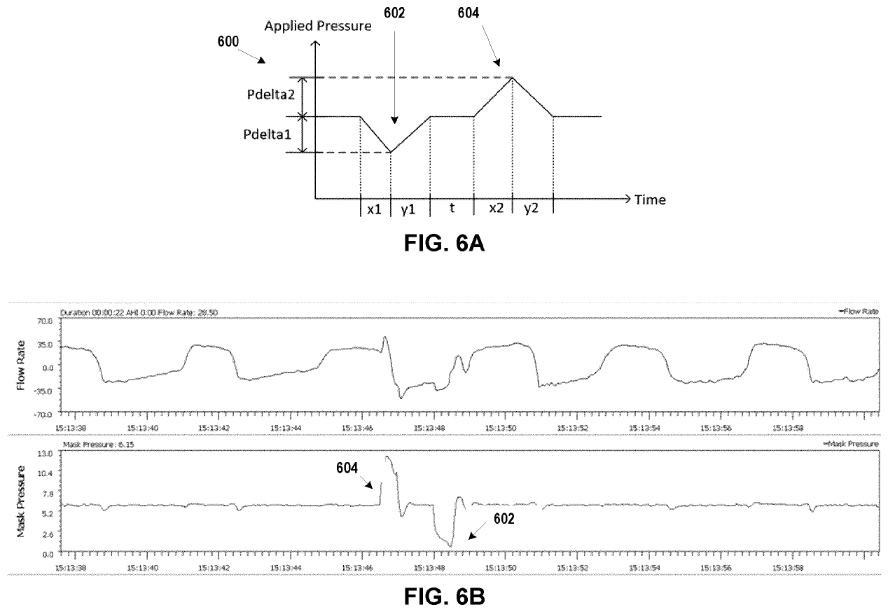

FIGS. 6A-6B show a combination of negative and positive pressure spikes routine 600. FIG. 6A is a graph of applied pressure to the user versus time. In the embodiment shown in FIG. 6A, a negative pressure spike 602 is first applied to the user. A negative pressure spike, such as the negative pressure spike 602, includes applying pressure with a negative slope for a period of time, and then subsequently applying pressure with a positive slope for a period of time to reach the initial pressure. In some embodiments, such as the example shown in FIG. 6A, after the negative pressure spike 602 is applied, the pressure may be held approximately constant for a period of time. In other embodiments, a positive pressure spike may be immediately applied after the negative pressure spike. FIG. 6A shows the application of a positive pressure spike 604 following the pressure being held constant. A positive pressure spike, such as the positive pressure spike 604, includes applying pressure with a positive slope for a period of time, and then subsequently applying pressure with a negative slope for a period of time to reach the initial pressure. In other embodiments, the routine 600 may involve applying a positive pressure spike followed by a negative pressure spike, with or without holding an approximately constant pressure in between. In the embodiment shown in FIG. 6, the pressure routine 600 involves applying two pressure spikes consecutively. In other embodiments, the routine 600 may involve applying any number of pressure spikes consecutively, varying their amplitude and/or varying the amount of time t in between successive spikes which may depend on the severity of the expected or currently occurring respiratory failure event.

FIG. 6B shows example flow rate and mask pressure versus time graphs demonstrating the application of the routine 600 on normal breathing with a PAP machine. In the example shown in FIG. 6B, the positive pressure spike 604 is applied to the user followed by holding the pressure constant, then the negative pressure spike 602 is applied to the user. A stable flow rate, amplitude, and pattern (or in other words, normal breathing) is maintained during and after the application of the pressure routine to the user.

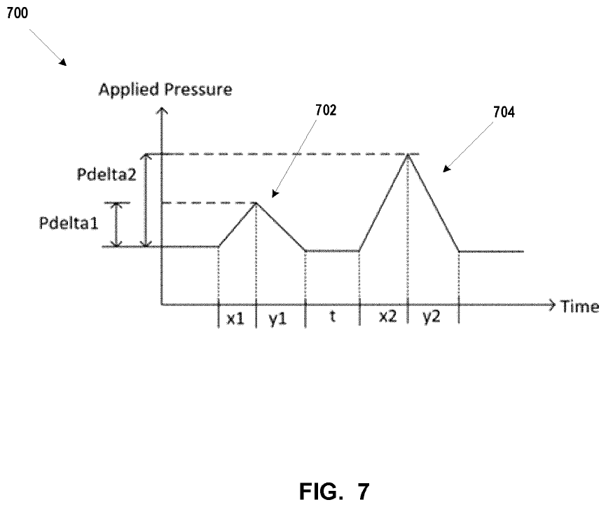

FIG. 7 shows a repeating variable amplitude pressure spikes routine 700. FIG. 7 is a graph of applied pressure to the user versus time. The pressure routine 700 includes applying consecutive positive pressure spikes, similar to positive pressure spike 604, or consecutive negative pressure spikes, similar to negative pressure spike 602. FIG. 7 shows an example embodiment of a first positive pressure spike 702 followed by a second positive pressure spike 704. The second positive pressure spike 704 reaches a higher amplitude (i.e., maximum pressure for positive spikes or minimum pressure for negative spikes) than the first positive pressure spike 702. In other embodiments, the pressure spikes may all reach the same amplitude or have different amplitudes such as progressively smaller amplitudes for each successive pressure spike. Other embodiments may also involve applying more than two pressure spikes consecutively, varying their amplitudes and/or varying the amount of time t in between successive spikes which may depend on the severity of the expected or currently occurring respiratory failure event.

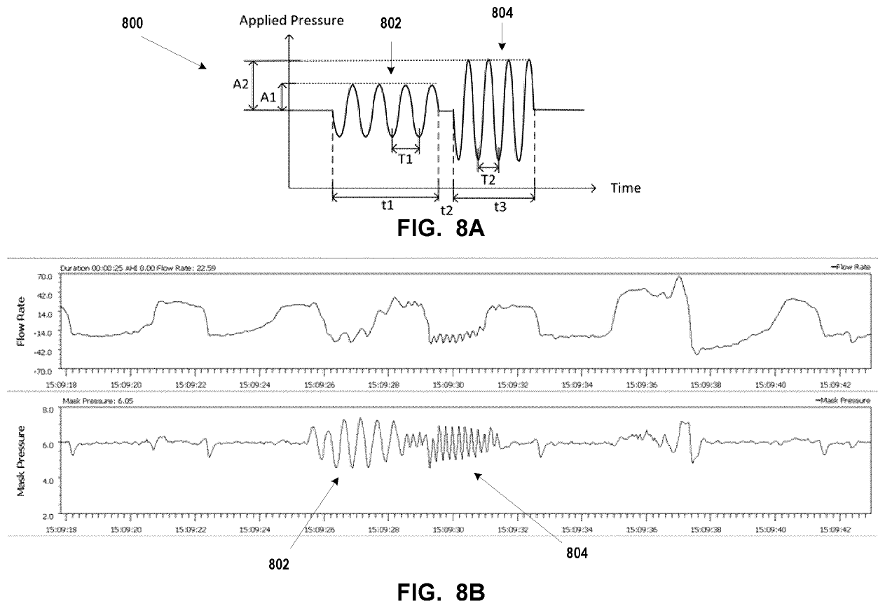

FIGS. 8A-8B show a combination of pressure sinusoids routine 800. This routine involves combinations of sinusoidal pressure waves with differing properties. FIG. 8A is a graph of applied pressure to the user versus time. For a first period of time, a first sinusoidal pressure wave 802 is applied to the user. Next, the pressure is held approximately constant for a period of time. In other embodiments, another sinusoidal pressure wave may be immediately applied after the first sinusoidal pressure wave 802. For the embodiment shown in FIG. 8A, a second sinusoidal pressure wave 804 is applied to the user after the pressure is held constant. In the embodiment shown in FIG. 8A, the second sinusoidal pressure wave 804 has a greater pressure amplitude (A2) and shorter period (T2) compared to the pressure amplitude (A1) and period (T1) of the first sinusoidal pressure wave 802. In other embodiments, the amplitude, period and duration of the sinusoidal waves may vary differently. In the embodiment shown in FIG. 8A, the pressure routine 800 involves applying two different sinusoidal waves consecutively. In other embodiments, the pressure routine 800 may involve applying any number of different sinusoidal waves consecutively, varying their amplitude, varying their period, varying their duration, and/or varying the amount of time t in between successive sinusoidal waves, which may depend on the severity of the expected or currently occurring respiratory failure event.

FIG. 8B shows flow rate and mask pressure versus time graphs demonstrating the application of the routine 800 on normal breathing with a PAP machine. The first sinusoidal pressure wave 802 is applied to the user followed by the second sinusoidal pressure wave 804. A stable flow rate, amplitude, and pattern (or in other words, normal breathing) is maintained during and after the application of the pressure routine to the user.

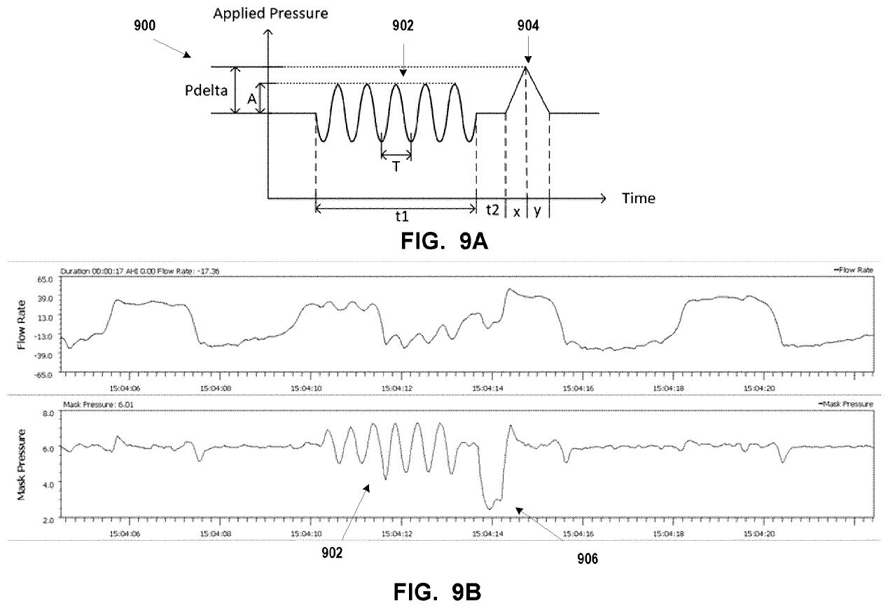

FIGS. 9A-9B show a combination of pressure sinusoids and pulses or spikes routine 900. This routine involves combinations of sinusoidal pressure waves with positive or negative pressure spikes or pulses. FIG. 9A is a graph of applied pressure to the user versus time. For a first period of time, a first sinusoidal pressure wave 902 is applied to the user. Next, the pressure is held approximately constant for a period of time. In other embodiments, a pressure pulse or spike may be immediately applied after the first pressure sinusoidal wave 902. For the embodiment shown in FIG. 9A, a positive pressure spike 904 is applied to the user after the pressure is held constant. The positive pressure spike 904 is similar to positive pressure spike 604. In other embodiments, a pressure pulse, similar to negative pressure pulse 402 or positive pressure pulse 404, is applied to the user. In other embodiments, the pressure spike applied to the user may be a negative pressure spike, similar to negative pressure spike 604. In other embodiments, the pressure pulse or spike may be applied first, followed by the sinusoidal wave. In the embodiment shown in FIG. 9A, the pressure routine 900 involves applying a single positive pressure spike 904 after the first sinusoidal pressure wave 902. In other embodiments, the pressure routine 900 may involve applying any number of pressure spikes or pulses that are positive or negative or a combination of both before or after the sinusoidal wave and/or varying the amount of time t in between successive waveforms, which may depend on the severity of the expected or currently occurring respiratory failure event.

FIG. 9B shows flow rate and mask pressure versus time graphs demonstrating the application of the routine 900 on normal breathing with a PAP machine. The first sinusoidal pressure wave 902 is applied to the user followed by a negative pressure spike 906, which is similar to negative pressure spike 604. A stable flow rate, amplitude, and pattern (or in other words, normal breathing) is maintained during and after the application of the pressure routine to the user.

Referring now to FIG. 10, shown therein is a flowchart of an example embodiment of a method 1000 for choosing and applying a pressure routine to a user of a breathing assistance device. The method may be performed by the controller 206 such as via processor 228, for example.

At step 1002, a processor, e.g., processor 228, obtains sensor data of one or more measurements that are made examples of which were provided in the description of the various sensors which may be used in the system 200.

At step 1004, the sensor data is used along with a determination/prediction method, as described previously, to determine whether a respiratory failure event is imminent or occurring. If a respiratory failure event is not imminent or occurring, the method 1000 returns to step 1002. If a respiratory failure event is determined to be imminent or occurring, the method 1000 moves to step 1006. This may be done using various techniques such as one or more of those described in U.S. Pat. Nos. 11,413,414B2, 11,633,560B2, 11,612,708B2, and/or PCT Patent Application No. PCT/CA2024/050324.

At step 1006, the processor determines which type of respiratory failure event may be imminent or occurring. For example, the processor 228 may execute software programs for a classifier that may classify the type of respiratory failure event. This may be done using various techniques such as one or more of those described in U.S. Pat. Nos. 11,413,414B2, 11,633,560B2, 11,612,708B2, and/or PCT Patent Application No. PCT/CA2024/050324. A respiratory failure event can include any event that deviates from normal breathing, such as a sleep apnea event, for example.

At step 1008, an appropriate pressure routine is selected and the control signal, e.g., control signal 212, is adjusted based according to the selected pressure routine to prevent the respiratory failure event or reduce its impact. The pressure routine may be selected based on the type of respiratory failure event that is predicted to occur imminently or is detected as currently occurring.

Selection of the pressure routine may be made in various manners. For example, selection of a pressure routine may be based on previously collected data from a large patient population showing that a certain pressure routine works best on a specific respiratory failure event. For instance, to activate the genioglossus muscle, a combination of negative and positive pressure spikes routine, e.g., routine 600, may be effective on most patients to prevent or reduce a respiratory failure. In another example, the pressure routine may be selected based on learnings from the same patient over several nights using various techniques such as one or more of those described in U.S. Pat. Nos. 11,633,560B2, 11,612,708B2, and/or PCT Patent Application No. PCT/CA2024/050324. For instance, while the combination of negative and positive pressure spikes routine, e.g., routine 600, may be effective on most users, for a certain user, the combination of pressure sinusoids and pulses or spikes routine, e.g., routine 900, may be more effective. In this case, the pressure routine 900 may be selected for that user.

In addition, in at least one embodiment, the pressure routine may be further personalized to a user. For example, the various parameters of the pressure routines, e.g., slope, frequency and amplitude, may be adjusted to best suit the specific user based on historical data. Accordingly, at step 1009, the various parameters of the pressure routine may be adjusted.

At steps 1010-1022, various pressure routines may be applied to the user. The routine that is applied depends on which one has been selected at step 1008. The parameters of the routine that is applied may depend on the adjustments made at step 1009.

At step 1010 a multi-variable slope pressure routine, e.g., routine 300, may be applied to the user.

At step 1012 a combination of negative and positive pressure pulses routine, e.g., routine 400, may be applied to the user.

At step 1014 a repeating pressure pulses routine, e.g., routine 500, may be applied to the user.

At step 1016 a combination of negative and positive pressure spikes routine, e.g., routine 600, may be applied to the user.

At step 1018 a repeating variable amplitude pressure spikes routine, e.g., routine 700, may be applied to the user.

At step 1020 a combination of pressure sinusoids routine, e.g., routine 800, may be applied to the user.

At step 1022 a combination of pressure sinusoids and pulses or spikes routine, e.g., routine 900, may be applied to the user.

In an alternative embodiment, it may be possible to select two or more different pressure routines.

After any of the pressure routines have been applied, the method 1000 returns to step 1002 and the method 1000 can be completed again for as long as the breathing assistance device is being used.

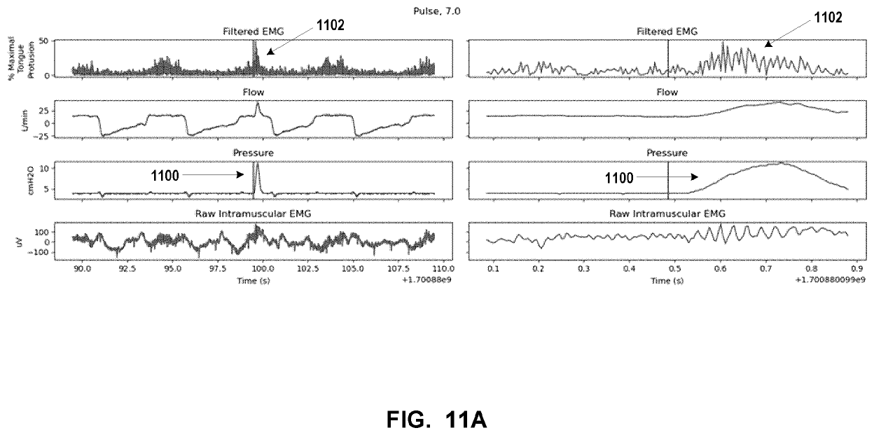

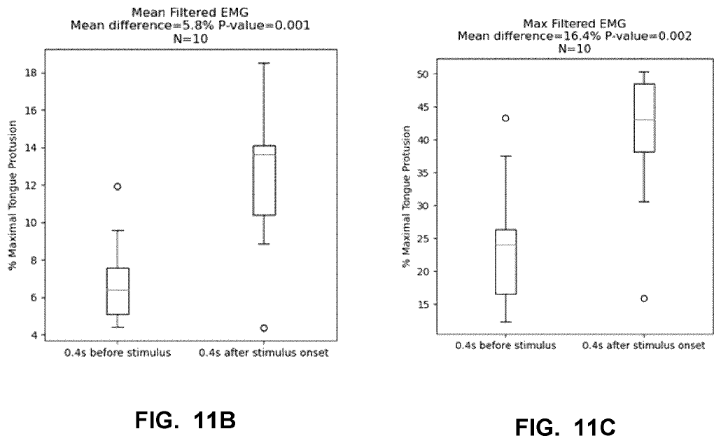

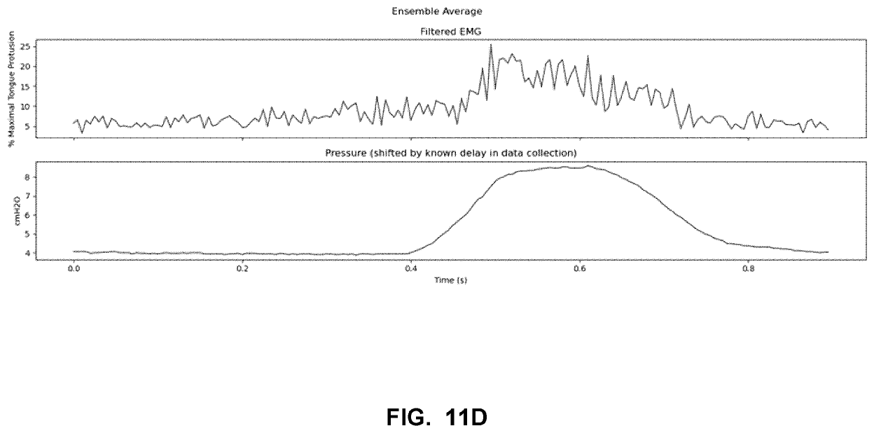

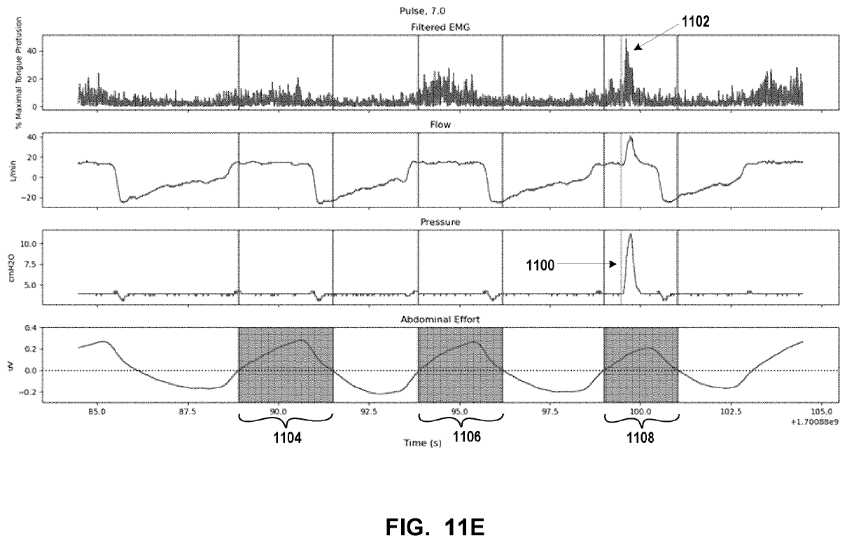

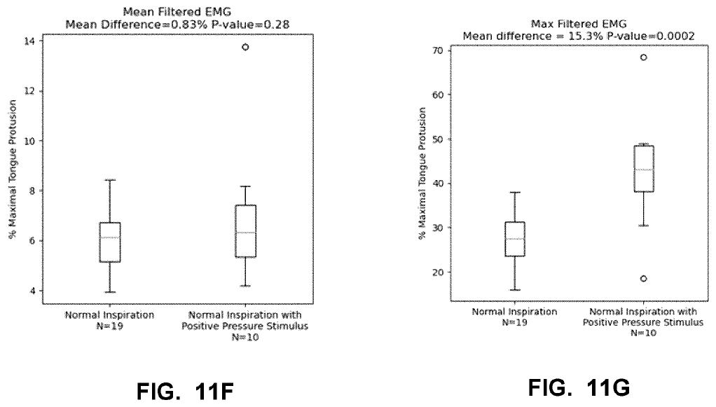

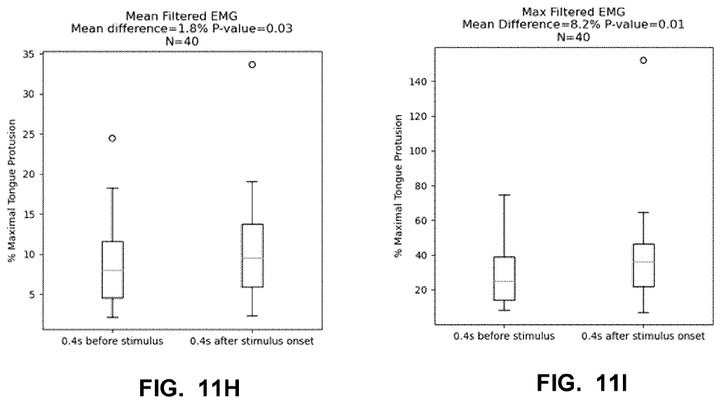

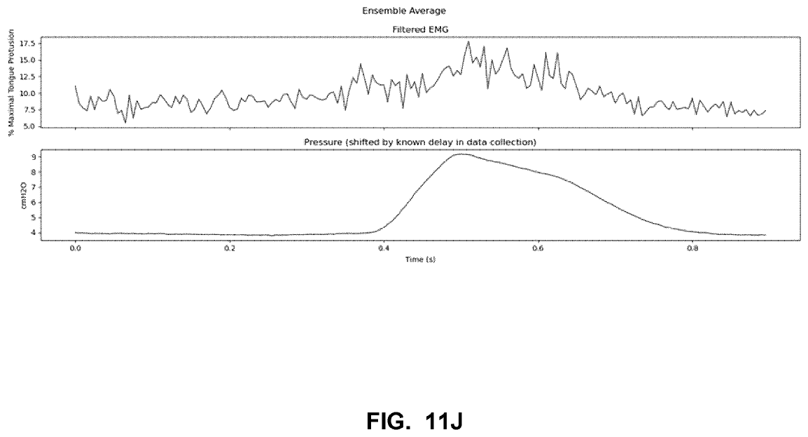

Reference is now made to FIG. 11A, which shows example filtered electromyography (EMG), flow, pressure and raw intramuscular EMG versus time graphs demonstrating the application of a pressure spike on normal breathing with a PAP machine. The raw and filtered EMG graphs demonstrates the EMG activity of the genioglossus of the user. The flow graph shows the airflow of the user. The pressure graph shows the measured pressure as applied by the PAP machine. The left graphs show a 20 second view, while the right graphs show a 0.8 second view. During the inspiration of the user, a positive pressure spike 1100 is applied to the user. As shown in the filtered EMG graph at 1102, the EMG activity increased immediately following the application of the pressure spike 1100, indicating an increase in muscle activity.

For one user, ten positive pressure spikes similar to spike 1100 were applied over a period of time. The pressure spikes ranged in amplitude from +4 cmH2O to +10 cmH2O and in duration from 0.1 to 0.3 seconds. FIGS. 11B and 11C show the mean filtered EMG and maximum filtered EMG, respectively, for the ten pressure spikes both at 0.4 seconds before the application of the pressure spike and at 0.4 seconds after the pressure spike. FIGS. 11B and 11C both show a statistically significant increase in the 0.4 seconds after the spike onset compared to the 0.4 seconds before the onset. A two-sided, paired t-test was used to show significance.

FIG. 11D shows filtered EMG and pressure graphs versus time for the ten spikes ensembled averaged together. FIG. 11D shows that the genioglossus EMG activity increases during the pressure spike.

FIG. 11E shows example filtered EMG, flow, pressure and abdominal effort graphs versus time with the application of the pressure spike 1100. FIG. 11E shows a comparison of two inhalation periods 1104, 1106 prior to the application of the pressure spike 1100 with the inhalation period 1108 where the spike 1100 was applied. As shown by FIG. 11E, the EMG increase 1102 seen after the application of the spike 1100 was not due to natural increases in genioglossus EMG activity during inhalation.

FIGS. 11F and 11G show the mean filtered EMG and max filtered EMG, respectively, for normal inspiration and normal inspiration with the positive pressure spike 1100. FIG. 11F shows a slight, nonsignificant increase in mean filtered EMG. FIG. 11G shows a significant increase in max filtered EMG. A 2-sided, independent t-test was used.

For one user, forty positive pressure spikes similar to spike 1100 were applied over a period of time. The pressure spikes ranged had an amplitude of at least +4 cmH2O. FIGS. 11H and 111 show the mean filtered EMG and maximum filtered EMG, respectively, for the forty pressure spikes both at 0.4 seconds before the application of the pressure spike and at 0.4 seconds after the pressure spike. FIGS. 11H and 111 both show a statistically significant increase in mean and maximum filtered EMG the 0.4 seconds after the spike onset compared to the 0.4 seconds before the onset. A two-sided, paired t-test was used to show significance. FIG. 11J shows filtered EMG and pressure graphs versus time for the forty spikes ensembled averaged together. FIG. 11J shows that the genioglossus EMG activity increases during the pressure spike.

FIGS. 11A-11J show that positive pressure spikes show the most genioglossus EMG activity increase when applied during the inhalation period, and result in higher overall activation of the genioglossus. Positive pressure spikes applied in any respiratory phase also show an increase in muscle activity. The user did not arouse due to any of the pressure maneuvers (i.e., applications of the pressure spikes).

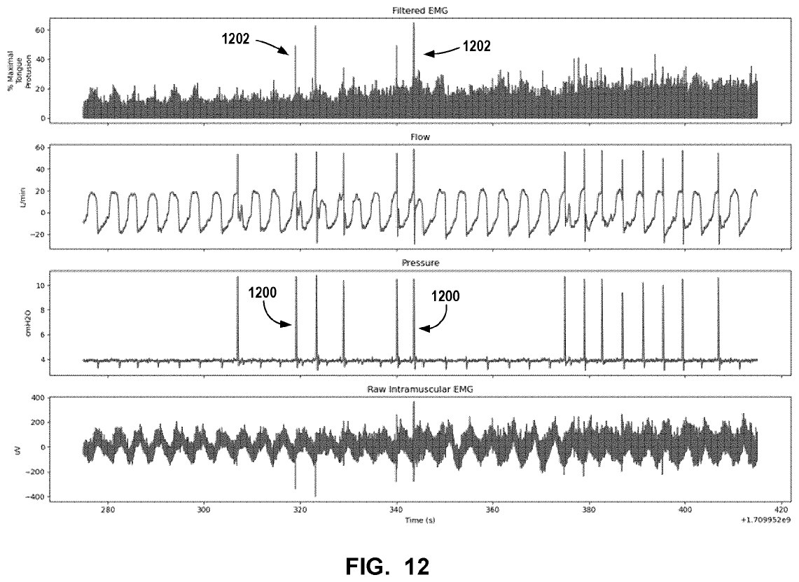

Reference is now made to FIG. 12, which shows example filtered EMG, flow, pressure and raw intramuscular EMG versus time graphs demonstrating the application of a repeating pressure pulse routine (e.g., routine 500) on normal breathing with a PAP machine. Several pressure pulses 1200 of varying amplitude (between approximately 5-7 cmH2O) were applied to the user in the inhalation phase. As shown in the filtered EMG graph at 1202, the EMG activity increased immediately following the application of the pressure pulses 1200, indicating an increase in muscle activity.

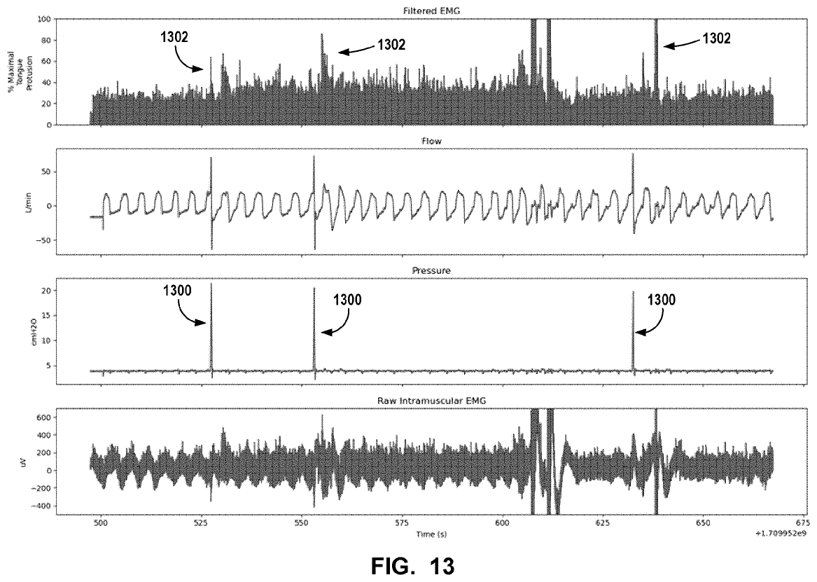

Reference is now made to FIG. 13, which shows a further example of filtered EMG, flow, pressure and raw intramuscular EMG versus time graphs demonstrating the application of a repeating pressure pulse routine (e.g., routine 500) on normal breathing with a PAP machine. Several pressure pulses 1300 of varying amplitude (around approximately 15 cmH2O) were applied to the user. As shown in the filtered EMG graph at 1302, the EMG activity increased immediately following the application of the pressure pulses 1300, indicating an increase in muscle activity.

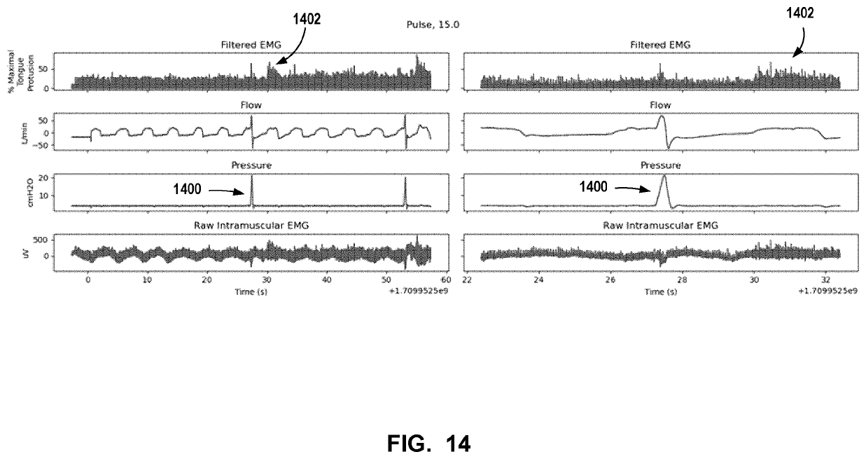

Reference is now made to FIG. 14, which shows a further example of filtered EMG, flow, pressure and raw intramuscular EMG versus time graphs demonstrating the application of a pressure spike on normal breathing with a PAP machine. The left graphs show a 60 second view, while the right graphs show a 10 second view. During the breathing of the user, a positive pressure spike 1400 with an amplitude of 15 cmH2O is applied to the user. As shown in the filtered EMG graph at 1402, the EMG activity increased on the next inhale following the application of the pressure spike 1400.

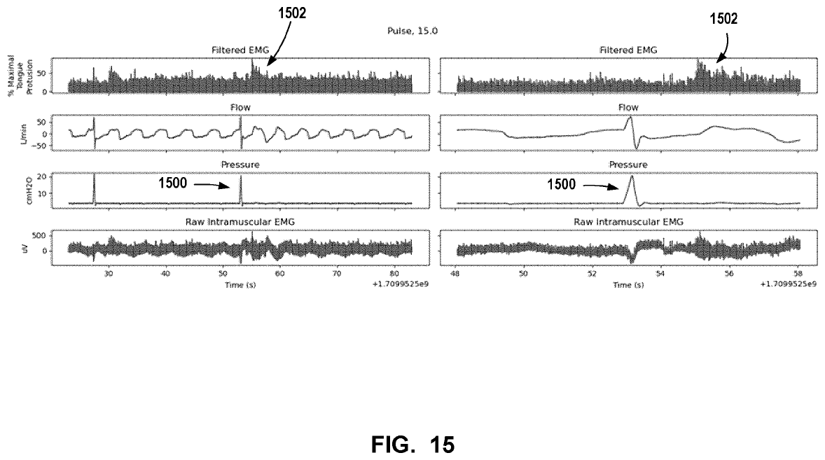

Reference is now made to FIG. 15, which shows a further example of filtered EMG, flow, pressure and raw intramuscular EMG versus time graphs demonstrating the application of a pressure spike on normal breathing with a PAP machine. The left graphs show a 60 second view, while the right graphs show a 10 second view. During the breathing of the user, a positive pressure spike 1500 with an amplitude of 15 cmH2O is applied to the user. As shown in the filtered EMG graph at 1502, the EMG activity increased on the next inhale following the application of the pressure spike 1500.

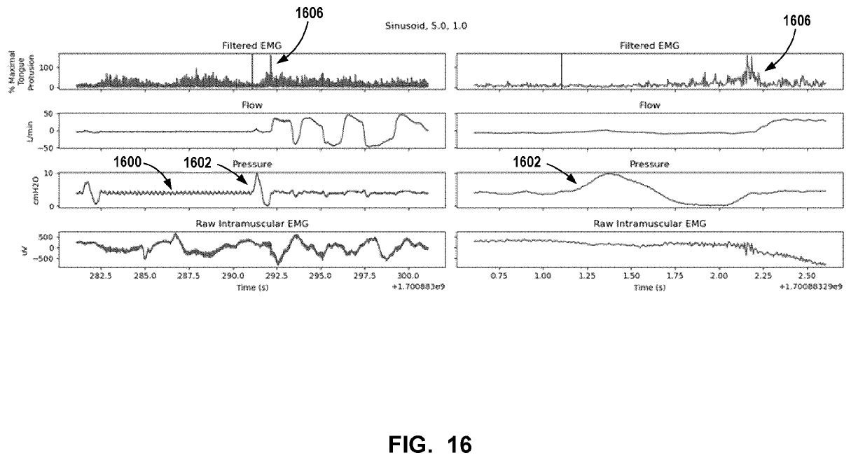

Reference is now made to FIG. 16, which shows example filtered EMG, flow, pressure and raw intramuscular EMG versus time graphs demonstrating the application of a combination sinusoidal routine (e.g., routine 800) on normal breathing with a PAP machine. The left graphs show a 20 second view, while the right graphs show a 2 second view. During an apnea event, a period of forced oscillation 1600 is applied to the user at 4 Hz, followed by a 1 Hz, 5 cmH20 amplitude sine wave 1602. The apnea ends after the application of the sine wave 1602, and EMG activity increases at 1606.

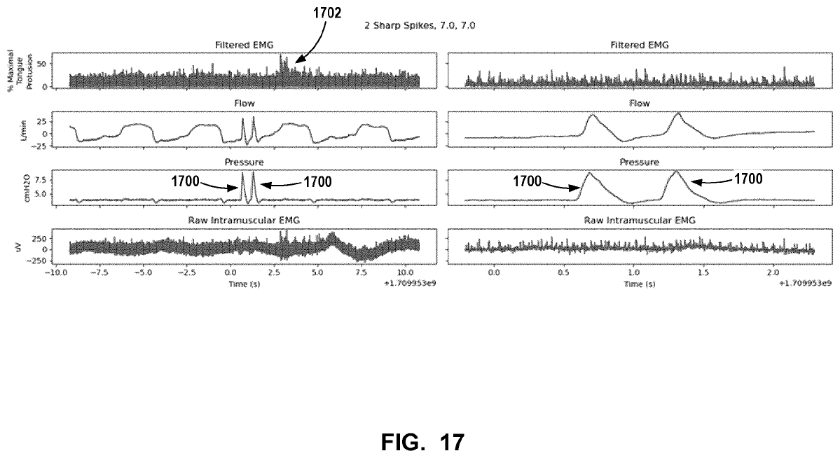

Reference is now made to FIG. 17, which shows an example of filtered EMG, flow, pressure and raw intramuscular EMG versus time graphs demonstrating the application of a repeating pressure spike routine (e.g., routine 700) on normal breathing with a PAP machine. Two positive pressure spikes 1700 with an amplitude of approximately +7 cmH2O were applied to the user. As shown in the filtered EMG graph at 1702, the EMG activity increased on the next inhale following the application of the pressure spikes 1700.

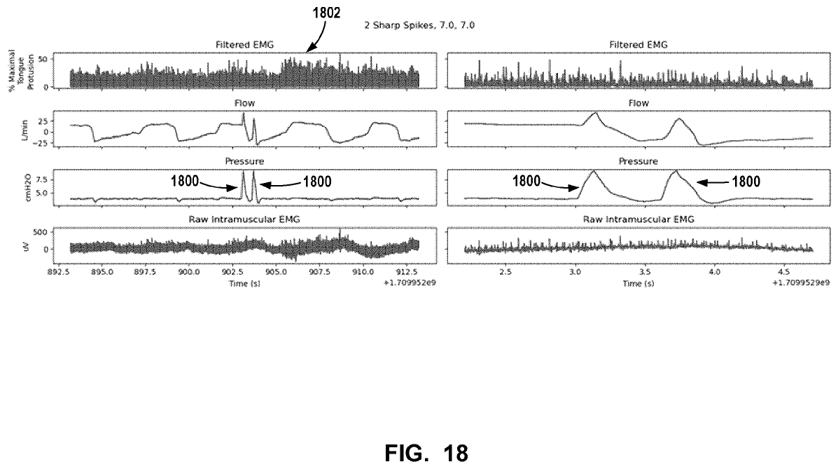

Reference is now made to FIG. 18, which shows a further example of filtered EMG, flow, pressure and raw intramuscular EMG versus time graphs demonstrating the application of a repeating pressure spike routine (e.g., routine 700) on normal breathing with a PAP machine. Two positive pressure spikes 1800 with an amplitude of approximately +7 cmH2O were applied to the user. As shown in the filtered EMG graph at 1802, the EMG activity increased on the next inhale following the application of the pressure spikes 1800.

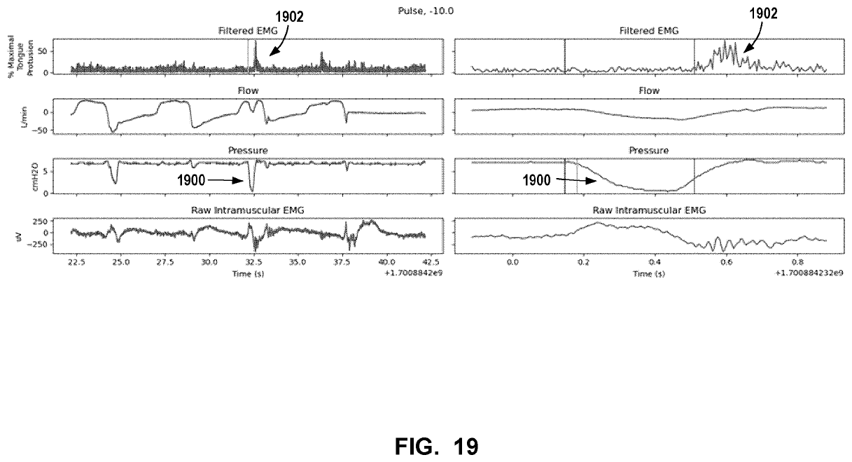

Reference is now made to FIG. 19, which shows an example of filtered EMG, flow, pressure and raw intramuscular EMG versus time graphs demonstrating the application of a negative pressure spike on normal breathing with a PAP machine. A negative pressure spike 1800 lowering to an amplitude of approximately 0 cmH2O was applied to the user. As shown in the filtered EMG graph at 1902, the EMG activity increased following the application of the negative pressure spike 1900.

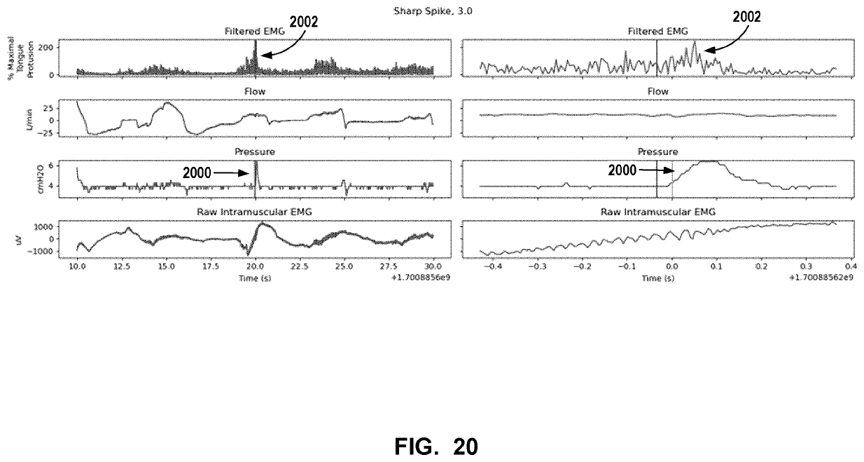

Reference is now made to FIG. 20, which shows an example of filtered EMG, flow, pressure and raw intramuscular EMG versus time graphs demonstrating the application of a positive pressure spike on normal breathing with a PAP machine. A positive pressure spike 2000 with an amplitude of approximately 3 cmH2O was applied to the user. As shown in the filtered EMG graph at 2002, the EMG activity increased following the application of the positive pressure spike 2000.

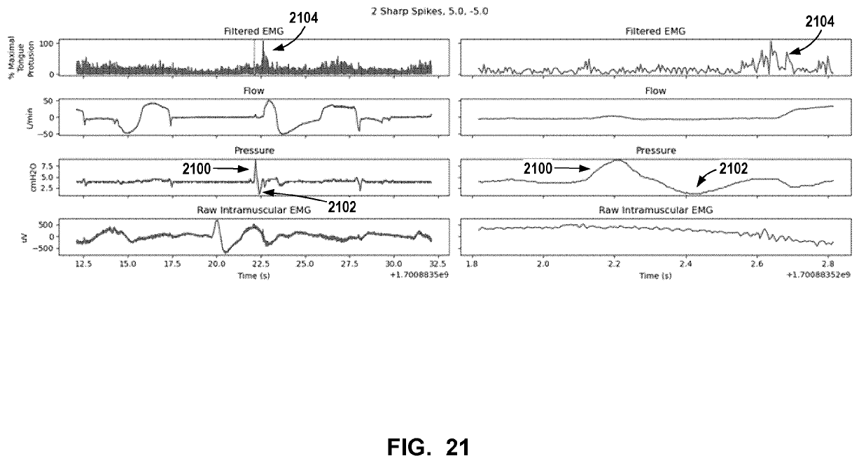

Reference is now made to FIG. 21, which shows an example of filtered EMG, flow, pressure and raw intramuscular EMG versus time graphs demonstrating the application of a combination negative and positive pressure spike routine (e.g., routine 600) on normal breathing with a PAP machine. A positive pressure spike 2100 with an amplitude of approximately +5 cmH2O was applied to the user, followed by a negative pressure spike 2102 lowering to an amplitude of 0cmH2O. As shown in the filtered EMG graph at 2104, the EMG activity increased following the application of the positive pressure spike 2100 and negative pressure spike 2102.

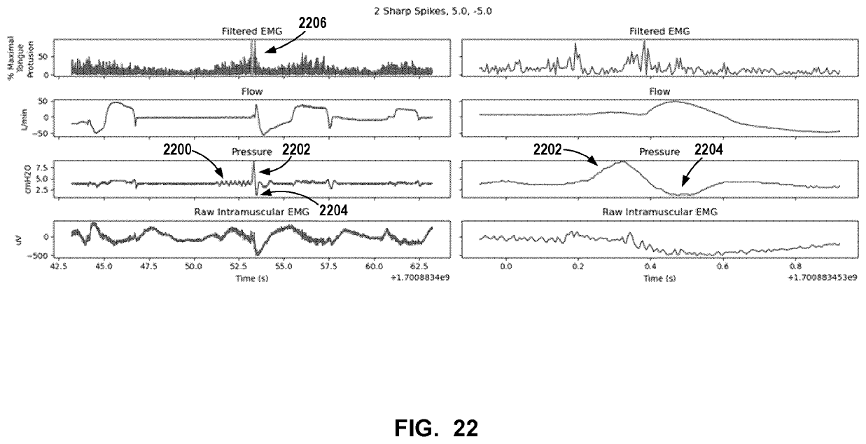

Reference is now made to FIG. 22, which shows an example of filtered EMG, flow, pressure and raw intramuscular EMG versus time graphs demonstrating the application of a combination sinusoidal pressure waves and negative and positive pressure spike routine (e.g., routine 900) on normal breathing with a PAP machine. During an apnea event, a period of forced oscillation 2200 is applied to the user at 4 Hz, followed by a +5 cmH2O amplitude positive pressure spike 2202 and a 0 cmH2O amplitude negative pressure spike 2204. The apnea ends after the application of the negative pressure spike 2204, and EMG activity increases at 2206.

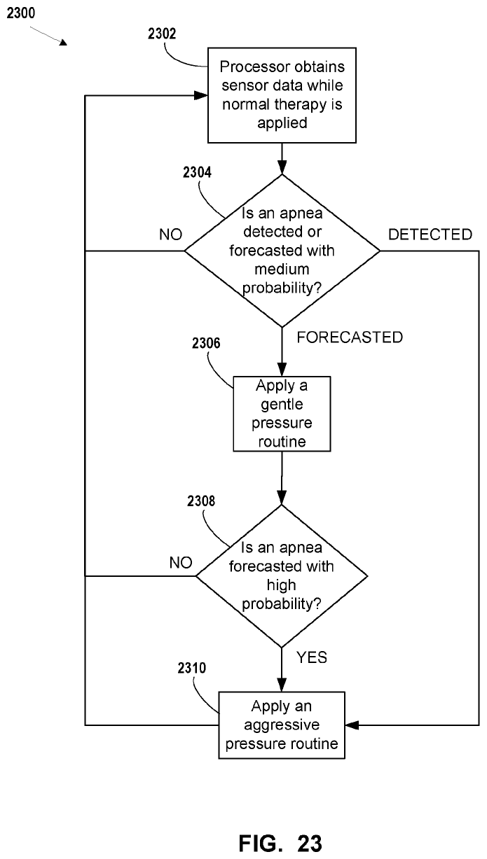

Referring now to FIG. 23, shown therein is a flowchart of an example embodiment of a method 2300 for choosing and applying a pressure routine to a user of a breathing assistance device. The method 2300 may be performed by the controller 206, such as via processor 228, for example.

At step 2302, a processor, e.g., processor 228, obtains sensor data of one or more measurements that are made, examples of which were provided in the description of the various sensors which may be used in the system 200. While this sensor data is obtained by the processor, normal therapy is applied to the user.

At step 2304, the sensor data is used along with a determination/prediction method, as described previously, to determine whether and with what probability a respiratory failure (i.e., apnea) is currently detected (i.e., occurring) or forecasted (i.e., imminent). This may be done using various techniques such as one or more of those described in U.S. Pat. Nos. 11,413,414B2, 11,633,560B2, and U.S. Pat. No. 11,612,708B2, and/or PCT Patent Application No. PCT/CA2024/050324. If an apnea is neither forecasted nor detected, the method 2300 returns to step 2302. If an apnea is detected with at least a medium probability (i.e., around at least about 60% to about 70% probability), the method 2300 moves to step 2310. If an apnea is forecasted with at least a medium probability, the method 2300 moves to step 2306.

At step 2306, a gentle pressure routine is applied to the user. A gentle pressure routine may be any pressure routine disclosed herein (e.g., routines 300, 400, 500, 600, 700, and 800), where the amplitude of the applied routine is relatively low. For example, a gentle pressure spike may have an amplitude of about 0 to about 5 cmH2O. As another example, an aggressive pressure spike may have an amplitude of greater than about 5 cmH2O. After applying the gentle pressure routine, the method 2300 moves to step 2308.

At step 2308, further sensor data is obtained by the processor and used to determine whether an apnea is forecasted with a high probability (i.e., around at least 70%). In other words, the data is used to determine whether the gentle pressure routine applied at step 2306 prevented the imminent apnea (e.g., imminent apnea may mean an apnea within about the next 60 seconds). If an apnea is no longer forecasted, the method 2300 returns to step 2302. If an apnea is forecasted with a high probability (e.g., a high probability may be from about 70% to about 100%), then the method 2300 moves to step 2310. The forecast may be made for up to the next approximately 60 seconds.

At step 2310, an aggressive pressure routine is applied to the use. An aggressive pressure routine may be any pressure routine disclosed herein (e.g., routines 300, 400, 500, 600, 700, and 800), where the amplitude of the applied routine is relatively high. For example, an aggressive pressure spike may have an amplitude of about 6 cmH20 to about 15 cmH2O.

In some embodiments, the aggressive pressure routine applied at step 2310 may be the same type of pressure routine as at step 2306 (e.g., both may be repeating variable amplitude pressure spikes routines) but a higher pressure is used (e.g., such as about 5 to about 15 cmH2O). In some embodiments, an aggressive pressure routine may have a pressure that is about 100% to about 300% higher than a gentle pressure routine. In other embodiments, the aggressive pressure routine applied at step 2310 may be a different type of pressure routine than at step 2306 (e.g., the gentle pressure routine may be a repeating pressure pulse routine, while the aggressive pressure routine may be a combination of pressure sinusoids routine, or vice versa). In either case, in at least one embodiment, the aggressive combination pressure routine may include a pressure amplitude/maximum of about 5 to about 15 cmH2O or higher, while the gentle combination pressure routines may have less than about 5 cmH2O in pressure peak/maximum/amplitude (regardless of if whether the waveforms include a sinusoid, a pulse, a spike or is positive or negative (e.g. about −15 cmH2O) The various parameters of the pressure routines, including the amplitude and type, may be chosen based on what will best suit the specific user, as well as historical data. In at least one embodiment, parameters other than amplitude may be varied to make the stimulus routine more aggressive or more gentle such as the length of time that the stimulus is applied or how many times a routine is repeated. As another example, when the pulse length lasts less than about 1 second this may be considered aggressive and when th pulse length lasts from about 1 seconds to about 20 seconds this may be considered gentle. In at least one embodiment, for stimulus routines including sinusoids or spikes, the amplitude may be increased/decreased to make the stimulus more/less aggressive.

After applying the aggressive pressure routine at step 2310, the method moves back to step 2302.

While the applicant's teachings described herein are in conjunction with various embodiments for illustrative purposes, it is not intended that the applicant's teachings be limited to such embodiments. On the contrary, the applicant's teachings described and illustrated herein encompass various alternatives, modifications, and equivalents, without generally departing from the embodiments described herein. For example, while the teachings described and shown herein may comprise certain elements/components and steps, modifications may be made as is known to those skilled in the art. For example, selected features from one or more of the example embodiments described herein in accordance with the teachings herein may be combined to create alternative embodiments that are not explicitly described. All values and sub-ranges within disclosed ranges are also disclosed. The subject matter described herein intends to cover and embrace all suitable changes in technology.

### REFERENCES

[1] N. M. Punjabi, “The Epidemiology of Adult Obstructive Sleep Apnea,” Proc. Am. Thorac. Soc., vol. 5, no. 2, pp. 136-143, Feb. 2008, doi: 10.1513/pats.200709-155MG.
[2] K. P. Grace, S. W. Hughes, and R. L. Horner, “Identification of the Mechanism Mediating Genioglossus Muscle Suppression in REM Sleep,” Am. J. Respir Crit. Care Med., vol. 187, no. 3, pp. 311-319, Feb. 2013, doi: 10.1164/rccm.201209-16540C.
[3] V. B. Fenik, T. Penzel, and A. Malhotra, “Editorial: Anatomy of Upper Airway and Neuronal Control of Pharyngeal Muscles in Obstructive Sleep Apnea,” Front. Neurol., vol. 10, 2019, Accessed: Dec. 21, 2022. [Online]. Available: https://www.frontiersin.org/articles/10.3389/fneur.2019.00733
[4] D. J. Eckert, A. S. Jordan, P. Merchia, and A. Malhotra, “Central Sleep Apnea: Pathophysiology and Treatment,” Chest, vol. 131, no. 2, pp. 595-607, Feb. 2007, doi: 10.1378/chest.06.2287.
[5] R. B. Fogel et al., “Genioglossal activation in patients with obstructive sleep apnea versus control subjects. Mechanisms of muscle control,” Am. J. Respir. Crit. Care Med., vol. 164, no. 11, pp. 2025-2030, Dec. 2001, doi: 10.1164/ajrcom.164.11.2102048.
[6] C. A. Edwards, A. Kouzani, K. H. Lee, and E. K. Ross, “Neurostimulation Devices for the Treatment of Neurologic Disorders,” Mayo Clin. Proc., vol. 92, no. 9, pp. 1427-1444, Sep. 2017, doi: 10.1016/j.mayocp.2017.05.005.
[7] S. Roy, “Therapy Update: Implanted Neurostimulators for Sleep Apnea,” Sleep Review, May 28, 2019. https://sleepreviewmag.com/sleep-treatments/therapy-devices/neurostimulators/neurostimulators-sleep-apnea/ (accessed Dec. 21, 2022).
[8] A. R. Schwartz, D. W. Eisele, A. Hari, R. Testerman, D. Erickson, and P. L. Smith, “Electrical stimulation of the lingual musculature in obstructive sleep apnea,” J. Appl. Physiol. Bethesda Md 1985, vol. 81, no. 2, pp. 643-652, Aug. 1996, doi: 10.1152/jappl.1996.81.2.643.

1. A method for stimulating one or more airway muscles of a user of a breathing assistance device, wherein the method is performed by a processor, the method comprising:
receiving sensor data from at least the user;
processing the sensor data to predict whether a respiratory failure event is imminent or to detect that the respiratory event is currently occurring when the user is using the breathing assistance device;
selecting one or more of air pressure routines to prevent the predicted respiratory failure event or to mitigate the respiratory failure event that is currently occurring;
adjusting the control signal of the breathing assistance device based on the selected one or more pressure routines; and
providing the control signal to the breathing assistance device to apply the selected one or more air pressure routines to the user.

2. The method of claim 1, wherein the one or more air pressure routines comprise at least two sequential waveforms that have different shapes or similar shapes with different characteristics.

3. The method of claim 1, wherein the one or more air pressure routines comprise varying between different slopes of applied pressure magnitude over time.

4. The method of claim 1, wherein the one or more air pressure routines comprise combinations of negative and positive pressure pulses.

5. The method of claim 1, wherein the one or more air pressure routines comprise repeating pressure pulses.

6. The method of claim 1, wherein the one or more air pressure routines comprise combinations of negative and positive pressure spikes.

7. The method of a claim 1, wherein the one or more air pressure routines comprise repeating pressure spikes of varying amplitude.

8. The method of claim 1, wherein the one or more air pressure routines comprise combinations of sinusoidal pressure waves with differing properties.

9. The method of claim 1, wherein the one or more air pressure routines comprise combinations of sinusoidal pressure waves and pressure spikes or pulses.

10. The method of claim 1, the method further including:
receiving subsequent sensor data from at least the user subsequent to the application of the selected one or more air pressure routines;
processing the subsequent sensor data to predict whether a respiratory failure event remains imminent when the user is using the breathing assistance device;
adjusting the control signal of the breathing assistance device to apply the selected air pressure routine at a higher amplitude; and
providing the control signal to the breathing assistance device to apply the selected one or more air pressure routines to the user at the higher amplitude.

11. A controller for controlling the operation of a breathing assistance device for stimulating one or more airway muscles of a user that uses the breathing assistance, wherein the controller comprises:
an input for receiving sensor data from at least the user;
a memory unit that comprises instructions for applying one or more air pressure routines during operation of the breathing assistance device;
a processor that is electronically coupled to the input to receive the sensor data, the processor being configured to generate a control signal for controlling the breathing assistance device by:
processing the sensor data to predict whether a respiratory failure event is imminent or is detect that the respiratory event is currently occurring when the user is using the breathing assistance device;
selecting one or more of the air pressure routines to prevent the predicted respiratory failure event or to mitigate the respiratory failure event that is currently occurring;
adjusting the control signal of the breathing assistance device based on the selected one or more pressure routines; and
providing the control signal to the breathing assistance device to apply the selected one or more air pressure routines to the user.

12. The controller of claim 11, wherein the controller is further configured to perform the method defined according to 2.

13. A system for stimulating the airway muscles of a user of a breathing assistance device, wherein the system comprises:
a breathing assistance device that generates an airflow to the user;
an entry element that is coupled to the breathing assistance device and is worn by the user to provide the airflow to the user during use; and
a breathing assistance device controller that is defined according to claim 11.

14. The system of claim 13, wherein the breathing assistance device comprises a high flow delivery device.

15. The system of claim 13, wherein the breathing assistance device comprises a gas delivery device including an oxygen delivery device.

16. The controller of claim 11, wherein the controller is further configured to perform the method defir coordin to claim 4.

17. The controller of claim 11, wherein the controller is further configured to perform the method defined according to claim 8.

18. The controller of claim 11, wherein the controller is further configured to perform the method defined according to claim 9.

19. The system of claim 13, wherein the controller is further configure to perform the method defined according to claim 4.

20. The system of claim 13, wherein the controller is further configured to perform the method defined according to claim 9.
